(19) United States
(12) Patent Application Publication
(10) Pub. No.: US 2026/0014250 A1
(43) Pub. Date: Jan. 15, 2026

(54) IMMUNOCONJUGATE

(71) Applicant: THE FRANCIS CRICK INSTITUTE LIMITED, London, Greater London (GB)

(72) Inventors: Caetano REIS E SOUSA, London, Greater London (GB); Oliver SCHULZ, London, Greater London (GB)

(21) Appl. No.: 18/997,334

(22) PCT Filed: Jul. 21, 2023
(86) PCT No.: PCT/EP2023/070303

§ 371 Date: Jan. 21, 2025

(30) Foreign Application Priority Data
Jul. 21, 2022 (GB) 2210674.4
Jan. 31, 2023 (GB) 2301356.8

(51) Int. Cl. A61K 39/385; A61K 39/00; A61P 37/04

(52) U.S. Cl. CPC A61K 39/385; A61P 37/04; A61K 2039/53; A61K 2039/55505; A61K 2039/572; A61K 2039/6031

(57) ABSTRACT

The present invention provides immunoconjugates comprising an antigen and an F-actin-binding moiety. These immunoconjugates promote the presentation of antigens. The invention also provides vaccines that comprise or encode conjugates tint allow antigens to be presented to the immune system. Related medical uses and methods of eliciting an immune response are also provided.

### CROSS REFERENCE TO RELATED APPLICATIONS

This application is a national stage application filed under 35 U.S.C. § 371 of PCT/EP2023/070303 filed Jul. 21, 2023, which claims the benefit of priority of GB Application No. 2210674.4 and GB Application No. 2301356.8, which are incorporated by reference in their entireties.

### REFERENCE TO SEQUENCE LISTING

The sequence listing submitted on Jan. 21, 2025, as an .XML file entitled “10480-026US1_ST26.xml” created on Jan. 17, 2025, and having a file size of 12,643 bytes is hereby incorporated by reference pursuant to 37 C.F.R. § 1.52(e)(5).

### FIELD OF THE INVENTION

The present invention relates to immunoconjugates, particularly agents that promote the presentation of antigens. The invention also provides vaccines that comprise or encode conjugates that allow antigens to be presented to the immune system. The invention further provides such a conjugate for use in a method of eliciting an immune response; methods of priming immune cells to attack a target cell; primed immune cells and nucleic acids and plasmids encoding the conjugate.

### BACKGROUND

Dendritic cells (DCs) are antigen presenting cells (APCs) which process antigen material for presentation purposes. Mature DCs present processed antigen epitopes on their surfaces via major histocompatibility complex class I (MHC 1) and class II (MHC II) molecules, thereby stimulating naïve CD8+ and CD4+ T-cells respectively. DCs also secrete IL-12 and interferon-γ (IFN-γ) to increase costimulatory factor production. To respond to malignancies, tumour-specific T-cells are activated through binding to MHC-peptide complex-T cell receptor and a costimulatory “signal 2”. These activated T-cells then differentiate into both long-lived memory T-cells and effector T-cells. Tumour-specific effector T-cells are then able to induce tumour killing through cytotoxicity and the production of effector cytokines. In addition, activated B-cells promote tumour apoptosis through antibody-dependent cellular cytotoxicity (ADCC) or complement-dependent cytotoxicity. The resulting immunogenic cell death releases tumour antigens and damage-associated molecular patterns (DAMPs). The released tumour antigens can be captured, processed, and presented again by APCs to induce polyclonal T-cell responses.

Type 1 conventional dendritic cells (cDC1) are indispensable for effective anti-tumour immunity (Wculek, 2020). This is, in part, attributable to their ability to acquire antigens from tumour cells, migrate to draining lymph nodes, and prime cancer-specific CD8+ T cells (Alloatti, 2017; Salmon, 2016; Theisen 2018). cDC1 cells also play an important role in infectious disease (Colin & Bigley, 2018). Both these responses require the presentation of antigens on MHC class I molecules, a process termed cross-presentation (XP). Although many antigen presenting cells (APC), such as type 2 cDC (cDC2), may be capable of performing XP per se, cDC1 appear specialised for this process in the context of cell-associated antigens.

The cytoskeleton plays a role in immunity and is composed of different protein filaments that provide mechanical support including microtubules, intermediate filaments and actin filaments. These are composed of different filamentous proteins, such as tubulin and actin. Actin, which forms highly dynamic filaments, allows cells to change shape. Various cells of the immune system, including macrophages, DCs and granulocytes utilise the cytoskeleton to generate large membrane protrusions. These can be used in phagocytosis, during which the coalescence of these protrusions form cytosolic vacuoles (Mylvaganam, 2021).

Other immune cells interact with the cytoskeleton in other ways. For example, cDC1 cells express high levels of the C-type lectin receptor DNGR-1 (also known as CLEC9A), which recognises filamentous actin (F-actin) exposed on necrotic cells (WO 2013/088136; Hand, 2015; Zhang, 2012; Ahrens 2012). In this way, necrotic cell debris, which is avidly internalised by cDC1, may act as a source of antigens for XP (Galluzzi, 2017). Notably, DNGR-1 expression is highly restricted to cDC1 in both mice and humans (Poulin, 2012) and acts as a receptor dedicated to XP of necrotic cell-associated antigens. Upon binding to F-actin via its C-type lectin domain (CTLD), DNGR-1 triggers SYK signalling, which causes rupture of ligand-containing phagosomes, release of antigenic material into the cDC1 cytosol, and its entry into the endogenous MHC class I presentation pathway (Sancho, 2009; Canton, 2021; WO2009/013484A1). Importantly, as F-actin exposure is associated with pathological cell death (e.g., necrosis) rather than apoptosis, DNGR-1 may act as a necrotic cell sensor to specifically couple recognition of tissue damage to the activation of a cytotoxic CD8+ T cell response. As such, DNGR-1 can play an important role in the priming of cytotoxic CD8+ T cells against cytopathic pathogens or tumours.

The activity of DNGR-1 is regulated by secreted gelsolin (sGSN), one of two abundant actin-binding proteins (the other being Gc globulin) present in plasma, that contribute to the removal of potentially pathological actin filaments released from or exposed by dying cells following tissue damage (Giampazolias, 2021). sGSN binds to F-actin in a Ca2+-dependent manner and severs the filaments for subsequent depolymerisation, which is facilitated by Ca2+-independent sequestering of monomeric G-actin by Gc globulin. In this way, sGSN can mask the activity of DNGR-1 by preventing binding to F-actin.

DNGR-1 itself is a type II transmembrane protein, with the extracellular C-terminal CTLD of DNGR-1 connected via a neck region to a transmembrane (TM) domain and subsequent hemITAM (hemi-immunoreceptor tyrosine-based activation motif)-containing N-terminal intracellular tail (Huysamen, 2008; Sancho, 2008). The hemITAM motif is required for Syk binding. Mice are known to have five different isoforms of DNGR-1, of which only two isoforms possess the entire ligand-binding domain and TM region. These isoforms are termed “long” (isoform 4) and “short” (isoform 1). Humans only have a single isoform, which corresponds to the “short” mouse isoform. The “long” mouse isoform is distinguished from the “short” mouse isoform and the human isoform by an extra exon which codes for an additional 26 amino acids in the neck region.

Vaccines are biological agents that elicit an immune response against a specific antigen. Different types of vaccines include inactivated vaccines, live-attenuated vaccines, toxoid vaccines, viral vector vaccines (such as adenovirus vector vaccines) and mRNA vaccines. Vaccines are often administered with an adjuvant, in order to increase the immunogenicity of the vaccine.

Viral vector vaccines use modified viruses as vaccine carriers. The innate immunogenicity of viruses helps to promote an effective immune response to the introduced antigen. While different viruses can be used, many adenovirus-based vaccines are currently being developed. Initially popular as a genetic delivery vector, the highly immunogenic properties of adenovirus make it an ideal candidate as a vaccine vector (Chang, 2021).

mRNA vaccines represent a safe vector to introduce nucleic acid encoded antigens. As mRNA does not interact with the genome, there is no risk of insertion events. They also offer greater flexibility during vaccine development that other vaccine types as the sequence information can be easily adapted (Schlake, 2012).

Traditionally, vaccines are used as a preventative measure against infectious disease. However, they can also be used to treat cancer. The aim of cancer vaccines is to stimulate an immune response to cancer-specific antigens. These include both preventative vaccines which can guard against tumour development and therapeutic vaccines which help treat cancer that has already been identified.

Cancer vaccines differ from traditional vaccines because they target endogenous tumour antigens with low immunogenicity. It is often difficult to elicit an effective immune response to tumour antigens, which include tumour-associated antigens (TAAs) and tumour-specific antigens (TSAs). TAAs include proteins that are present in normal tissue but are aberrantly expressed in tumours (overexpression, different subcellular localization). By comparison, TSAs are only found in cancerous tissue (Morse, 2021). They are also referred to as neoantigens. TAAs have been the focus of clinical trials for many years, with limited success. As TAAs are expressed in non-malignant tissues, vaccines targeting TAAs come with the risk of vaccine-induced autoimmune toxicity. As TSAs are only found in cancerous tissue, they show increased immunogenicity as compared to TAAs.

Many types of vaccines are known in the art (Liu, 2022). Cancer vaccines typically rely on CD8+ cytotoxic T cell mediated cellular immunity for maximum efficacy.

Virus based vaccines use viruses as vectors to deliver nucleic acids. Peptide-based vaccines use known or predicted tumour antigen epitopes. These are often less immunogenic and require a combination with adjuvants to enhance their immunogenicity.

Nucleic acid-based vaccines, also known as genetic vaccines, include both DNA and RNA vaccines which encode tumour antigens. Non-viral nucleic acid-based vaccines typically use lipid-based systems, such as liposomes, to deliver the nucleic acids to cells. Virus-based vaccines, and nucleic acid-based vaccines more generally, are intrinsically immunostimulatory, helping to initiate the innate immune response (Morse, 2021).

DNA cancer vaccines are closed circular DNA plasmids encoding TAAs or immunomodulatory molecules to induce tumour-specific responses. These DNA molecules need to enter the cell nucleus to initiate transcription. By comparison mRNA vaccines, which are synthesized in vitro to encode antigens, enter the cytoplasm and translate and express antigens directly.

Therefore, mRNA antigen production is instantaneous and efficient. DNA vaccines need an extra step to go into the cell nucleus, leading to a lower immune response than mRNA vaccines. However, once plasmid DNA enters the nucleus, a single plasmid DNA can produce multiple mRNA copies, producing more antigens than a single mRNA molecule. Finally, DNA vaccines have a potential risk of insertion mutations. However, mRNA vaccines have no risk of insertion and integration into the genome.

The present invention has been devised in light of the above considerations.

### SUMMARY OF THE INVENTION

An underlying principle of immunotherapy is targeting antigens that are either not present in healthy, normal cells, or are expressed to a lesser degree. In the case of cancer, this means targeting neo-antigens, tumour-associated antigens (TAAs), and tumour-specific antigens (TSAs). In the case of infectious diseases, this means targeting antigens that are only present in the infectious agent, and which are not expressed by the host cell. Currently, most immunotherapy approaches have focused on targeting antigens which are already ‘visible’ to the immune system. Here, the inventors sought to utilise the cytoskeleton to expand the number of antigens that antigen presenting cells (APCs) can access and mount an immune response against, by conjugating an antigen to a moiety that binds to a cytoskeleton component, specifically, F-actin. Through such conjugation, the inventors sought to improve the retention of the antigen at the site of cell death, thus increasing the likelihood the antigen will be taken up by APCs and lead to priming of an immune response against the antigen.

The inventors have previously shown that in sGSN^{-/—} mice, cross-presentation of Ovalbumin (OVA) may be improved by conjugating OVA to the F-actin binding peptide Lifeact (Giampazolias (2021)) and the fluorescent protein mCherry. These conjugates find use in studies investigating the impact of sGSN in XP. However, these conjugates were injected into tumour cells and subsequently injected into mice. As such, the conjugates are not acting as a vaccine: instead, the LA-OVA-mCherry conjugate of this system acts as a neoantigen, causing the immune system to be primed against the cells that express the LA-OVA-mCherry conjugate, rather than priming the immune system against other target cells that express OVA.

In a first aspect, the invention provides a vaccine comprising a nucleic acid that encodes a conjugate comprising an antigen and an F-actin-binding moiety. In some embodiments, the nucleic acid is an RNA molecule, such as mRNA. In some embodiments, the nucleic acid is DNA.

In a related aspect, the invention provides a vaccine comprising a conjugate comprising an antigen and an F-actin-binding moiety.

In embodiments where the vaccine comprises a nucleic acid (e.g. mRNA) that encodes the conjugate, the nucleic acid may be delivered to a receiver cell. This receiver cell may be, for example, a muscle cell. The receiver cell will then express the conjugate in the cytosol, where it can bind F-actin. In some embodiments, the receiving cell undergoes immunogenic cell death (ICD). Following permeabilization of the receiver cell by the way of, for example, necrotic cell death, the conjugate will be retained at the site of vaccination. For example, the conjugate may bind to F-actin exposed on the surface of a necrotic cell. In some cases the receiver cell is the necrotic cell to which the conjugate binds. This can promote internalization by an APC. For example, conjugating an antigen to an F-actin binding component enables the conjugate to indirectly bind to DNGR-1 via F-actin exposed on necrotic cells. As described above, following phagocytosis DNGR-1 triggers SYK signalling, which causes rupture of ligand-containing phagosomes, causing release of antigenic material into the cDC1 cytosol, and its entry into the endogenous MHC class I presentation pathway (Sancho, 2009; Canton, 2021; WO2009/013484A1). Therefore, the vaccine primes the immune system against the antigen of the conjugate.

Immune response to vaccination can involve release of the vaccinated antigen(s) via immunogenic cell death (ICD, Marichal et al, 2011). Such vaccine ICD could facilitate exposure of F-actin bound antigen conjugate and promote immune response against the antigen. ICD is defined by cell death that results in chronic exposure of damage-associated molecular patterns (DAMPs) which stimulates the immune system. Whereas necrosis is the most well-known form of ICD, other forms of cell death, such as ferroptosis or pyroptosis, can also activate immune response (Tang et al, 2020). Furthermore, immune mediated cell killing itself has also been shown to be immunogenic (Minute et al, 2020).

By necrotic cell it is meant a cell that has undergone (and is consequently dead) or is undergoing (but may not yet be dead) cell death whereby the membrane has been permeabilised or ruptured and/or there is extracellular exposure of the cytoskeleton. For example, the cell death mechanism may be necrosis.

In some embodiments, the vaccine is a non-viral vaccine. In other embodiments, the vaccine is a viral vaccine.

In some embodiments, the immune response comprises XP, MHC class II presentation, MHC Class II expression, presentation of exogenous antigens by MHC Class II, XP of MHC Class I antigens, phagocytosis of macromolecular complexes comprising an antigen, induction of type I interferon (IFN) expression, and/or delivery of macromolecular complexes comprising an antigen to the cytosol of an immune cell. In some embodiments, the immune response is elicited by a dendritic cell. In some embodiments, the dendritic cell is a type I conventional dendritic cell (cDC1). In some embodiments, the dendritic cell is a type II conventional dendritic cell (cDC2). In some embodiments, the immune response comprises activation of a T cell response. In some embodiments, the T cell response is a CD8+ T cell response. In some embodiments, the antigen is cross-presented on an MHC class I molecule. In some embodiments, the T cell response is a CD4+ T cell response. In some embodiments, the antigen is presented on an MHC class II molecule. In some embodiments, the immune response comprises activation of a B cell response. In some embodiments, internalisation of the conjugate is mediated by binding of the cytoskeleton component exposed on the necrotic cell to DNGR-1 on the surface of the immune cell.

In some embodiments, the F-actin-binding moiety is LifeAct, actinin, anillin, ezrin, fascin, filamin, F-tractin, an aptamer, an affimer, or a functional fragment thereof, or an F-actin binding protein selected from Table 1 (or a functional fragment thereof). As used herein, the term “F-actin binding moiety” does not include actin-destabilising proteins that sever F-actin filaments. More specifically, the term “F-actin binding moiety” does not include fully functional actin-destabilising proteins that sever F-actin filaments. However, the term “F-actin binding moiety” may include the binding fragment of such actin-destabilsing protein, wherein said binding fragment does not retain actin-destablising activity. For example, sGSN and cofilin are not encompassed in their fully functional form. However a non-destabilsing F-actin binding fragment is encompassed. In some embodiments, the F-actin binding moiety binds to actin filaments. In some embodiments, the F-actin binding moiety binds to actin bundles.

In some embodiments wherein the construct is delivered as a nucleic acid, the F-actin binding moiety must be active in the reducing environment of the cytosol, i.e. no disulphide bridges. The skilled person can readily design the construct to locate to the cytosol, e.g. by simply omitting signal peptides from the construct. Thus, in some embodiments, the conjugate does not comprise a signal peptide. In other embodiments, the conjugate is secreted and binds F-actin following cell death or other permeabilsation, in which case the F-actin binding moiety does not need to be active in reducing environments and so can contain disulphide bridges etc.

In embodiments where the conjugate is secreted, or in aspects where the vaccine comprises a (protein) conjugate, the F-actin-binding moiety may be selected from the group consisting of an aptamer, an affimer, a Fab fragment, a single chain Fc fragment (ScFv), a single domain antibody (sdAb), a variable domain (FV), an antibody, and an F-actin-binding peptide. In some embodiments, the F-actin binding peptide is specific for F-actin. In some embodiments, the F-actin binding peptide has greater specificity for F-actin relative to G-actin. In some embodiments, the specificity for F-actin is between 2- and 100-fold greater than for G-actin. In some embodiments, the specificity for F-actin is between 2- and 10-fold greater than for G-actin. In some embodiments, the specificity for F-actin is at least 2-fold greater than for G-actin. In some embodiments, the specificity for F-actin is at least 5-fold greater than for G-actin. In some embodiments, the specificity for F-actin is at least 10-fold greater than for G-actin. In some embodiments, the specificity for F-actin is at least 100-fold greater than for G-actin. Specificity is explicitly intended to encompass affinity and/or avidity. In some embodiments, the F-actin binding peptide binds both F-actin and G-actin. For the avoidance of doubt, the invention does not encompass conjugates comprising an F-actin-binding peptide that binds to G-actin but does not bind to F-actin. In some embodiments, the antibody is an anti-actin antibody. In some embodiments, the anti-actin antibody only binds F-actin and does not bind G-actin. In some embodiments, the F-actin-binding moiety is an affimer, wherein the affimer is specific for F-actin. In some embodiments, the affimer is an anti-actin affimer. In some embodiments, the affimer only binds F-actin and does not bind G-actin. In some embodiments, the affimer binds to actin filaments. In some embodiments, the affimer binds to actin bundles. In some embodiments, the affimer comprises the consensus sequence ATGVRAVPGNENSLEIEELARFAVDEHNKKENALLEFVRWKAKEQXXXXXXXXXTMYYLTLEAKDGGKK KLYEAKVWVKXXXXXXXXXNFKELQEFKPVGDA (SEQ ID NO: 1), wherein the first XXXXXXXXX region encodes a first loop (Loop 1) and the second XXXXXXXXX region encodes a second loop (Loop 2), and wherein the affimer specifically binds F-actin, as measured by plate-bound F-actin ELISA (Lopata, 2018) or co-sedimentation assay with F-actin (Lopata, 2018; Sugizaki, 2021). In some embodiments, the first and second XXXXXXXXX regions are randomised. In some embodiments, Loop 1 is IDLTEWQDR (SEQ ID NO: 5). In some embodiments, Loop 2 is PEPIHSHHS (SEQ ID NO: 6). In some embodiments, the affimer is the affimer described as Affimer 6 in Tiede (2014). In some embodiments, the affimer comprises the sequence of SEQ ID NO: 2 (Affimer 6). In some embodiments, the affimer displays at least about 80%, 81%, 82%, 83%, 84% 85%, 86%, 87%, 88%, 89%, 90%, 91%, 92%, 93%, 94%, 95%, 96%, 97%, 98% or 99% amino acid sequence identity with SEQ ID NO: 2 (Affimer 6). In some embodiments, Loop 1 is WFDDEYDWI (SEQ ID NO: 7). In some embodiments, Loop 2 is DYAATDLYW (SEQ ID NO: 8). In some embodiments, the affimer is the affimer described as Affimer 14 in Tiede (2014). In some embodiments, the affimer comprises the sequence of SEQ ID NO: 3 (Affimer 14). In some embodiments, the affimer displays at least about 80%, 81%, 82%, 83%, 84% 85%, 86%, 87%, 88%, 89%, 90%, 91%, 92%, 93%, 94%, 95%, 96%, 97%, 98% or 99% amino acid sequence identity with SEQ ID NO: 3 (Affimer 14). In some embodiments, Loop 1 is WEDFQTHWE (SEQ ID NO: 9). In some embodiments, Loop 2 is DVGQLLSGI (SEQ ID NO: 10). In some embodiments, the affimer is the affimer described as Affimer 24 in Tiede (2014). In some embodiments, the affimer comprises the sequence of SEQ ID NO: 4 (Affimer 24). In some embodiments, the affimer displays at least about 80%, 81%, 82%, 83%, 84% 85%, 86%, 87%, 88%, 89%, 90%, 91%, 92%, 93%, 94%, 95%, 96%, 97%, 98% or 99% amino acid sequence identity with SEQ ID NO: 4 (Affimer 24).

In aspects where the vaccine comprises a nucleic acid that expresses the conjugate, the F-actin-binding moiety may be selected from the group consisting of LifeAct, actinin, anillin, ezrin, fascin, filamin, F-tractin. Further F-actin-binding proteins are presented in Table 1, below. These proteins, or F-actin-binding fragments thereof, can be used as the F-actin-binding moiety.

In aspects where the conjugate is delivered as a peptide, the conjugate does not require a receiver cell to be expressed. Instead, the conjugate can simply bind to F-actin released by the vaccination induced cell death.

In some embodiments, the antigen comprises an endogenous antigen. In some embodiments, the endogenous antigen comprises a tumour-associated antigen (TAA) or a tumour-specific antigen (TSA). In preferred embodiments, the antigen is a surface polypeptide, i.e. it is expressed on the surface of a tumour.

In some embodiments, the antigen comprises an exogenous antigen. In some embodiments, the exogenous antigen comprises a viral, bacterial or fungal antigen. In preferred embodiments, the antigen is a surface polypeptide, i.e. it is expressed on the surface of a virus, bacterium or fungus.

In some embodiments, the antigen is linked to the F-actin-binding moiety by a linker, preferably a peptide linker. The skilled person can readily choose a linker that enables the F-actin-binding moiety to retain its ability to bind F-actin. In some embodiments, the linker is a peptide linker. In some embodiments, the peptide linker comprises the amino acid sequence IEGR. In some embodiments, the peptide linker comprises multiple glycine and/or serine residues. In some embodiments, the peptide linker comprises the amino acid sequence GGGGSGGGGS (SEQ ID NO: 11). In some embodiments, the peptide linker comprises the amino acid sequence ARTGGGGSGGGGSDI (SEQ ID NO:12). In some embodiments, the linker comprises a serine-rich and/or glycine-rich peptide. In some embodiments, the linker is 5 to 100, 10 to 90, 20 to 80, 30 to 70, 40 to 60, or 45 to 50 amino acids in length. In some embodiments, the linker is 10 amino acids in length. In some embodiments, the linker is 15 amino acids in length. Preferably, the linker is non-immunogenic in humans.

In some embodiments, the antigen is positioned N-terminal relative to the F-actin-binding moiety. In other embodiments, the antigen is positioned C-terminal relative to the F-actin-binding moiety.

In some embodiments, the conjugate is not inked to a fluorescent protein.

In some embodiments, the vaccine comprises, or is administered to a subject in combination with, an adjuvant. In some embodiments, the adjuvant is Alum.

In some embodiments, the vaccine comprises, or is administered to a subject in a composition comprising a pharmaceutically acceptable carrier. For example, the pharmaceutically acceptable carrier may comprise cationic lipids such as 1,2-di-O-octadecenyl-3-trimethylammonium-propane (DOTMA); Upofectin (a combination of DOTMA and 1,2-dioleoyl-sn-glycero-3-phosphoethanolamine (DOPE)); or 1,2-dioleoyl-3-trimethylammonium-propane (DOTAP). The pharmaceutically acceptable carrier may comprise ionizable lipids. The composition may be a liposome nanoparticle (LNP).

In some embodiments, the vaccine is an mRNA vaccine, meaning that the vaccine comprises an mRNA molecule that encodes the conjugate and can express it in a cell. The mRNA vaccine may comprise a nonreplicating mRNA or a self-amplifying mRNA. Preferably, the mRNA comprises a 5′ cap, 5′ UTRs, an open reading frame (ORF), 3 UTRs and a poly(A) tail. The mRNA can include certain features to increase efficacy such as a Cap-0, a Cap-1 or a Cap-2 at the 5 end (Uang et al, 2021). The mRNA may comprise an anti-reverse cap analog (ARCA). The 5′ UTR may include one or more structures or functional units such as a stem loop structure, an upstream open reading frame (uORF), an internal ribosome entry site (IRES), a cis element that can be bound by RNA binding proteins, a 7-methyl-guanosine cap (m7G), a hairpin-like secondary structures (hairpin), a Zip code, a cytoplasmic polyadenylation element (CPE), and/or a polyadenylation signal AAUAAA. The 3′ UTR may include stabilizing elements and may exclude GU-rich elements (GREs). The poly(A) tail may be at least 60 nucleotides in length, at least 80 nucleotides in length, at least 100 nucleotides in length, or at least 120 nucleotides in length.

In some embodiments, the subject has cancer, and the vaccine is an anti-cancer vaccine.

In some embodiments, the subject has an infectious disease, and the vaccine is an infectious disease vaccine.

In some embodiments, the vaccine is administered in combination with another vaccine. In some embodiments, the vaccine is a therapeutic vaccine. In some embodiments, the vaccine is a preventative vaccine.

In a second aspect, the invention provides an agent for use in a method of eliciting an immune response in a subject in need thereof, wherein the agent comprises a nucleic acid encoding a conjugate comprising an antigen conjugated to a F-actin-binding moiety and wherein the conjugate is not inked to a fluorescent protein. In some embodiments, the nucleic acid is RNA, e.g. mRNA. In other embodiments, the nucleic acid is DNA. In some embodiments, the agent is a non-viral vector. In other embodiments, the agent is a viral vector.

In a related aspect, the invention provides a peptide conjugate comprising an antigen conjugated to a F-actin-binding agent, for use in a method of eliciting an immune response in a subject in need thereof, wherein the conjugate is not inked to a fluorescent protein.

In some embodiments, the F-actin-binding peptide is selected from the group consisting of LifeAct, actinin, anillin, ezrin, fascin, filamin, F-tractin, an aptamer, an affimer, or an F-actin binding protein selected from Table 1 and a functional fragment thereof.

In some embodiments, the agent is a peptide. In these embodiments, the conjugate does not require a receiver cell to be expressed. Instead, the conjugate can immediately bind to F-actin exposed within necrotic cell debris, which may be generated by accidental cell death or by regulated cell death (RCD).

In some embodiments, the antigen comprises an endogenous antigen. In some embodiments, the endogenous antigen comprises a tumour-associated antigen (TAA) or a tumour-specific antigen (TSA).

In some embodiments, the antigen comprises an exogenous antigen. In some embodiments, the exogenous antigen comprises a viral, bacterial or fungal antigen.

In some embodiments, the antigen is inked to the F-actin-binding agent by a linker.

In some embodiments, the agent is administered to a subject in a vaccine. In some embodiments, the vaccine is a therapeutic vaccine. In some embodiments, the vaccine is a preventative vaccine.

In some embodiments, the agent is administered to a subject in combination with an adjuvant. In some embodiments, the adjuvant is Alum.

In some embodiments, the agent is administered to a subject in combination with a pharmaceutically acceptable carrier.

In some embodiments, the subject has cancer, and the immune response comprises an anti-cancer immune response.

In some embodiments, the subject has an infectious disease, and the immune response comprises an anti-infectious disease immune response.

In some embodiments, the agent is administered in combination with a vaccine.

In a third aspect, the invention provides a method of priming a lymphocyte to attack a target cell that expresses an antigen, comprising delivering an agent to a receiver cell, wherein the agent comprises a nucleic acid that expresses a conjugate comprising the antigen conjugated to a F-actin-binding moiety in the receiver cell; wherein the conjugate binds to F-actin that is exteriorised following receiver cell death; and wherein the conjugate is then recognised by an antigen presenting cell (APC) which subsequently presents the antigen to the lymphocyte, thus priming the lymphocyte to recognise the antigen on the target cell.

In a related aspect, the invention provides a method of priming a lymphocyte to attack a target cell that expresses an antigen, comprising delivering an agent to a receiver cell, wherein the agent comprises a conjugate comprising the antigen conjugated to a F-actin-binding moiety; wherein the conjugate binds to F-actin that is exteriorised following receiver cell death; and wherein the conjugate is then recognised by an antigen presenting cell (APC) which subsequently presents the antigen to the lymphocyte, thus priming the lymphocyte to recognise the antigen on the target cell. In some embodiments, the nucleic acid is RNA, e.g. mRNA. In other embodiments, the nucleic acid is DNA. In some embodiments, the agent is a non-viral vector. In other embodiments, the agent is a viral vector

In a fourth aspect, the invention provides an agent use in a method of priming a lymphocyte to attack a target cell that expresses an antigen in a subject, the method comprising delivering the agent to a receiver cell, wherein the agent comprises a nucleic acid that expresses a conjugate comprising the antigen and an F-actin-binding moiety in the receiver cell; wherein the conjugate binds to F-actin that is exteriorised following receiver cell death; and wherein the conjugate is then recognised by an antigen presenting cell (APC) which subsequently presents the antigen to the lymphocyte, thus priming the lymphocyte to recognise the antigen on the target cell. In a related aspect, the invention provides an agent use in a method of priming a lymphocyte to attack a target cell that expresses an antigen in a subject, the method comprising delivering the agent to a receiver cell, wherein the agent comprises a conjugate comprising the antigen and an F-actin-binding moiety in the receiver cell; wherein the conjugate binds to F-actin that is exteriorised following receiver cell death; and wherein the conjugate is then recognised by an antigen presenting cell (APC) which subsequently presents the antigen to the lymphocyte, thus priming the lymphocyte to recognise the antigen on the target cell.

In some embodiments, the immune cell is a dendritic cell. In some embodiments, the dendritic cell is a type I conventional dendritic cell (cDC1).

In some embodiments, priming of the immune cell leads to activation of a T cell response. In some embodiments, the T cell response is a CD8+ T cell response. In some embodiments, the antigen is cross-presented on an MHC class I molecule. In some embodiments, the T cell response is a CD4+ T cell response. In some embodiments, the antigen is presented on an MHC class II molecule. In some embodiments, priming of the immune cell leads to activation of a B cell response.

In some embodiments, the contacting step comprises delivering nucleic acid encoding the conjugate into the receiver cell, expression of the conjugate by the receiver cell, and release of the conjugate upon permeabilisation of the receiver cell.

In some embodiments, the nucleic acid is mRNA or DNA. In some embodiments, the mRNA is viral mRNA. In some embodiments, the DNA is viral DNA.

In some embodiments, the agent comprises a peptide.

In some embodiments, the receiving cell undergoes immunogenic cell death (ICD).

In some embodiments, the F-actin-binding agent is an F-actin-binding agent.

In embodiments where the conjugate is secreted, or in aspects where the agent comprises a (protein) conjugate, the F-actin-binding moiety may be selected from the group consisting of an aptamer, an affimer, a Fab fragment, a single chain Fc fragment (ScFv), a single domain antibody (sdAb), a variable domain (FV), an antibody, and an F-actin-binding peptide. In some embodiments, the antibody is an anti-actin antibody. In some embodiments, the anti-actin antibody only binds F-actin and does not bind G-actin. In some embodiments, the F-actin-binding moiety is an affimer, wherein the affimer is specific for F-actin. In some embodiments, the affimer is an anti-actin affimer. In some embodiments, the affimer only binds F-actin and does not bind G-actin. In some embodiments, the affimer binds to actin filaments. In some embodiments, the affimer binds to actin bundles. In some embodiments, the affimer comprises the consensus sequence ATGVRAVPGNENSLEIEELARFAVDEHNKKENALLEFVRVVKAKEQXXXXXXXXXTMYYLTLEAKDGGKK KLYEAKVWVKXXXXXXXXXNFKELQEFKPVGDA (SEQ ID NO: 1), wherein the first XXXXXXXXX region encodes a first loop (Loop 1) and the second XXXXXXXXX region encodes a second loop (Loop 2), and wherein the affimer specifically binds F-actin, as measured by plate-bound F-actin ELISA (Lopata, 2018) or co-sedimentation assay with F-actin (Lopata, 2018; Sugizaki, 2021). In some embodiments, the first and second XXXXXXXXX regions are randomised. In some embodiments, Loop 1 is IDLTEWQDR (SEQ ID NO: 5). In some embodiments, Loop 2 is PEPIHSHHS (SEQ ID NO: 6). In some embodiments, the affimer is the affimer described as Affimer 6 in Tiede (2014). In some embodiments, the affimer comprises the sequence of SEQ ID NO: 2 (Affimer 6). In some embodiments, the affimer displays at least about 80%, 81%, 82%, 83%, 84% 85%, 86%, 87%, 88%, 89%, 90%, 91%, 92%, 93%, 94%, 95%, 96%, 97%, 98% or 99% amino acid sequence identity with SEQ ID NO: 2 (Affimer 6). In some embodiments, Loop 1 is WFDDEYDWI (SEQ ID NO: 7). In some embodiments, Loop 2 is DYAATDLYW (SEQ ID NO: 8). In some embodiments, the affimer is the affimer described as Affimer 14 in Tiede (2014). In some embodiments, the affimer comprises the sequence of SEQ ID NO: 3 (Affimer 14). In some embodiments, the affimer displays at least about 80%, 81%, 82%, 83%, 84% 85%, 86%, 87%, 88%, 89%, 90%, 91%, 92%, 93%, 94%, 95%, 96%, 97%, 98% or 99% amino acid sequence identity with SEQ ID NO: 3 (Affimer 14). In some embodiments, Loop 1 is WEDFQTHWE (SEQ ID NO: 9). In some embodiments, Loop 2 is DVGQLLSGI (SEQ ID NO: 10). In some embodiments, the affimer is the affimer described as Affimer 24 in Tiede (2014). In some embodiments, the affimer comprises the sequence of SEQ ID NO: 4 (Affimer 24). In some embodiments, the affimer displays at least about 80%, 81%, 82%, 83%, 84% 85%, 86%, 87%, 88%, 89%, 90%, 91%, 92%, 93%, 94%, 95%, 96%, 97%, 98% or 99% amino acid sequence identity with SEQ ID NO: 4 (Affimer 24).

In some embodiments, the F-actin-binding peptide is selected from the group consisting of LifeAct, actinin, anillin, ezrin, fascin, filamin, F-tractin, an aptamer, an affimer, and a functional fragment thereof, or an F-actin binding protein selected from Table 1 and a functional fragment thereof.

In some embodiments, the antigen comprises an endogenous antigen. In some embodiments, the endogenous antigen comprises a tumour-associated antigen (TAA) or a tumour-specific antigen (TSA).

In some embodiments, the antigen comprises an exogenous antigen. In some embodiments, the exogenous antigen comprises a viral, bacterial or fungal antigen.

In some embodiments, the antigen is linked to the F-actin-binding moiety by a linker.

In some embodiments, the conjugate is not inked to a fluorescent protein.

In some embodiments, the conjugate is administered to the biological sample in combination with an adjuvant. In some embodiments, the adjuvant is Alum.

In some embodiments, the conjugate is administered to the biological sample in combination with a pharmaceutically acceptable carrier.

In some embodiments, the method is an in vivo method and comprises administering the agent to a subject in need thereof.

In some embodiments, the method is an ex vivo method and comprises priming the immune cell in vitro.

In some embodiments, the immune cell is obtained from a subject in need thereof. In some embodiments, the method further comprises harvesting the primed immune cell and administering the primed immune cell to the subject in need thereof.

In some embodiments, the subject has cancer. In some embodiments, the subject has an infectious disease.

In some embodiments, the method is an in vitro method. In some embodiments, the method further comprising harvesting the primed immune cell.

In a fourth aspect, the invention provides a primed immune cell obtained by the method of the third aspect.

In a fifth aspect, the invention provides a primed immune cell of the fourth aspect, for use as a vaccine.

In a sixth aspect, the invention provides a nucleic acid encoding the conjugate of any of the previous aspects.

In a seventh aspect, the invention provides a plasmid for making the conjugate of any of the previous aspects.

In an eighth aspect, the invention provides a method of treatment comprising administering a conjugate as described herein to a subject in need thereof.

In a ninth aspect, the invention provides the use of a conjugate as described herein for the manufacture of a medicament for the treatment of cancer.

In a tenth aspect, the invention provides the use of a conjugate as described herein for the manufacture of a medicament for the treatment of an infectious disease.

The invention includes the combination of the aspects and preferred features described except where such a combination is clearly impermissible or expressly avoided.

### SEQUENCES

### Affimer Consensus Sequence

The amino acid sequence of an affimer consensus sequence is set forth in SEQ ID NO: 1:

(SEQ ID NO: 1)
ATGVRAVPGNENSLEIEELARFAVDEHNKKENALLEFVRVVKAKEQXXX
XXXXXXTMYYLTLEAKDGGKKKLYEAKVWVKXXXXXXXXXNFKELQEFK
PVGDA

### Affimer 6

The amino acid sequence of Affimer 6 is set forth in SEQ ID NO: 2:

(SEQ ID NO: 2)
ATGVRAVPGNENSLEIEELARFAVDEHNKKENALLEFVRVVKAKEQIDL
TEWQDRTMYYLTLEAKDGGKKKLYEAKVWVKPEPIHSHHSNFKELQEFK
PVGDA

### Affimer 14

The amino acid sequence of Affimer 14 is set forth in SEQ ID NO: 3:

(SEQ ID NO: 3)
ATGVRAVPGNENSLEIEELARFAVDEHNKKENALLEFVRVVKAKEQWFD
DEYDWITMYYLTLEAKDGGKKKLYEAKVWVKDYAATDLYWNFKELQEFK
PVGDA

### Affimer 24

The amino acid sequence of Affimer 24 is set forth in SEQ ID NO: 4:

(SEQ ID NO: 4)
ATGVRAVPGNENSLEIEELARFAVDEHNKKENALLEFVRVVKAKEQWED
FQTHWETMYYLTLEAKDGGKKKLYEAKVWVKDVGQLLSGINFKELQEFK
PVGDA

### SUMMARY OF THE FIGURES

Embodiments and experiments illustrating the principles of the invention will now be discussed with reference to the accompanying figures in which:

FIG. 1. Lifeact (LA) peptide fusion targets OVA to F-actin and increases retention of OVA in HeLa cells following UV treatment. (A) Schematic illustration of the two OVA fusion constructs. The Lifeact (LA) peptide enables LAOVA to bind to F-actin when expressed in cells, whereas the three point mutations in mutated (mu)LAOVA prevent F-actin binding (Belyy, 2020) (data not shown) leading to cytoplasmic expression of muLAOVA. (B) Transduced or untransfected control HeLa cells were subjected to UV treatment, cultured overnight and then stained directly with an anti-His-tag mAb. Overlay histograms represent fluorescence profile of His-tagged fusion proteins (LAOVA or muLAOVA; right graph) or RFP (co-expressed protein encoded by the sleeping beauty plasmid; left graph). Numbers shown indicate GeoMean fluorescence intensity.

FIG. 2. Cells expressing OVA targeted to F-actin are cross-presented more efficiently by cDC1 In a DNGR-1-dependent manner. (A, B) Comparison of irradiated (UV) LAOVA and muLAOVA expressing HeLa cells to stimulate OT-I (A) or OT-II (B) T cells following presentation of dead cell-associated antigen by MutuDC. (C) Cross-presentation of LAOVA cells is strongly reduced in the presence of anti-DNGR-1 antibody (1F6) compared to cultures containing isotype control antibody (MAC49). (D) Comparison of DNGR-1-deficient Mutu DC reconstituted with either wildtype (C9A/KO) or double tryptophan mutant (2W/KO) DNGR-1 for their ability to cross-present irradiated (UV) LAOVA HeLa cells to OT-I T cells. (A-C) Necrotic cells were added at various doses as indicated to Mutu DC and pre-activated OT-I (A, C, D) or pre-activated OT-II co-cultures (B). Graphs show concentration of IFN-γ in the supernatant (SN) of overnight cultures. Plotted data represent mean±SD of duplicate wells. Data are representative of 3 (A, C) or 2 (B, D) independent experiments.

FIG. 3. Targeting OVA to F-actin in donor cells promotes cross-priming of OVA-specific CD8⁺ T cells in vivo. Induction of OVA-specific CD8 T cell responses following immunisation with UV-treated LAOVA or muLAOVA cells mixed with poly I:C (25 μg/mouse). (A) Schematic of the experimental set-up and analysis of OVA-specific CD8 T cells in immunised mice. Data shown are (B) percentage of IFN-γ⁺ CD44⁺ double-positive cells of total CD8 T cells and (C) absolute number of double positive cells.

FIG. 4. Lack of secreted gelsolin further increases cross-priming of OVA-specific CD8⁺ T cells in response to immunisation with LA-OVA cells. Induction of OVA-specific CD8 T cell responses following immunisation of wildtype (C57Bl6) or secreted gelsolin-deficient (sGSN^{-/-}) mice with UV-treated LAOVA or muLAOVA cells mixed with poly I:C (25 μg/mouse). Data shown are (A) percentage of IFN-g⁺ CD44⁺ double-positive cells of total CD8 T cells or (B) OVA-tetramer⁺ CD44⁺ double-positive cells of total CD8 T cells.

FIG. 5. Equal expression of Affimer-OVA fusion proteins in HeLa cells. (A) Schematic illustration of the two affimer-OVA fusion constructs. Through fusion with the actin-specific affimer 6, OVA gains the ability to bind to F-actin when expressed in cells, whereas OVA fused to a control affimer does not bind to F-actin (Lopata, 2018; Sugizaki, 2021). (B) Intra-cellular staining of HeLa cells stably transfected with affimer 6-OVA or control affimer-OVA as well as untransfected cells (negative control). Cells were fixed with PFA, permeabilised and stained with antibodies against His tag or OVA. Overlay histograms for His tag (left graph) and OVA (right graph) show fluorescence intensity profiles of affimer 6-OVA (top), control affimer-OVA (middle) and untransfected cells (bottom). Numbers shown indicate GeoMean fluorescence intensity.

FIG. 6. Cells expressing OVA anchored to F-actin via an Affimer (Affimer 6-OVA cells) are cross-presented more efficiently compared to control affimer-OVA cells. Comparison of (A) irradiated (UV) affimer 6-OVA and control affimer-OVA expressing HeLa cells and (B) necrotic LAOVA and affimer 6-OVA cells for their ability to be cross-presented to OT-I T cells by Mutu DC. (A, B) Necrotic cells were added at various doses as indicated to Mutu DC and pre-activated OT-I co-cultures. Graphs show concentration of IFN-γ in the SN of overnight cultures. Plotted data represent mean±SD of triplicate wells. Data are representative of 2 independent experiments.

FIG. 7. Antigen retention and phagosomal rupture may favour cross-presentation of neoantigens associated with the actin cytoskeleton. Schematic illustration of processing of cytoplasmic versus F-actin associated antigen by cDC1 following uptake of dead cell material. Antigens that remain associated with the actin-cytoskeleton are more likely to be internalised compared to cytosolic antigen, which drains away more easily following loss of membrane integrity. Furthermore, F-actin binding to DNGR-1 triggers phagosomal rupture, which may promote phagosome-to-cytosol release of antigen-F-actin complexes considered to be the rate-limiting step in the cross-presentation pathway.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Vaccine

A vaccine” is a composition of matter suitable for administration to a human or animal subject that is capable of eliciting a specific immune response, e.g., against a pathogen. The vaccine may be a therapeutic vaccine or a preventative (prophylactic) vaccine. The vaccine may be a nucleic acid vaccine.

The vaccine may be a viral vaccine, such as an adenovirus vaccine. Cell therapies or “cell-based vaccines” are excluded from the definition of the vaccine of the present invention.

The vaccine may be a cancer vaccine.

Nucleic acids

Besides RNA and DNA, modified nucleic acids may also be used in this invention. The nucleic acid may comprise a backbone comprising ribonucleic acid (RNA), deoxyribonucleic acid (DNA), DNA phosphorothioate, RNA phosphorothioate, 2′-0-methyl-oligoribonucleotide and 2′-0-methyl-oligodeoxyribonucleotide, 2′-0-hydrocarbyl ribonucleic acid, 2′-0-hydrocarbyl DNA, 2′-0-hydrocarbyl RNA phosphorothioate, 2′-0-hydrocarbyl DNA phosphorothioate, 2′-F-phosphorothioate, 2′-F-phosphodiester, 2′-methoxyethyl phosphorothioate, 2-methoxyethyl phosphodiester, deoxy methylene(methylimino) (deoxy MMI), 2′-0-hydrocarbyl MMI, deoxy-methylphos-phonate, 2′-0-hydrocarbyl methylphosphonate, morpholino, 4′-thio DNA, 4′-thio RNA, peptide nucleic acid, 3′-amidate, deoxy 3′-amidate, 2′-0-hydrocarbyl 3-amidate, locked nucleic acid, cyclohexane nucleic acid, tricycle-DNA, 2′fluoro-arabino nucleic acid, N3′-P5′ phosphoroamidate, carbamate inked, phosphotriester inked, a nylon backbone modification or mixtures of the aforementioned backbones.

### Antigens

The antigen may be any protein or fragment thereof against which it is desirable to raise an immune response, in particular a CTL response, but also a Th17 response or a Treg response. These may include antigens associated with, expressed by, displayed on, or secreted by cells against which it is desirable to stimulate a CTL response, including cancer cells and cells containing intracellular pathogens or parasites. For example, the antigen may be, or may comprise, an epitope peptide from a protein expressed by an intracellular pathogen or parasite (such as a bacterial protein, a viral protein or a fungal protein) or from a protein expressed by a cancer or tumour cell. Thus, the antigen may be a tumour-specific antigen. The term “tumour-specific” antigen should not be interpreted as being restricted to antigens from solid tumours, but to encompass antigens expressed specifically (or preferentially) by any cancerous, transformed or malignant cell. The antigen may be a tumour-associated antigen (TAA).

The antigen may be a necrotic cell-associated antigen. By necrotic cell we mean a cell that has undergone necrosis, or a cell that is in the process of necrosis. A necrotic cell-associated antigen may be necrotic cell debris (cellular components released from a necrotic cell during endosomal or phagosomal rupture). The necrotic cell-associated antigen may be an antigen that is not F-actin.

### F-Actin Binding Peptides

The F-actin binding peptide may be an FABP selected from Table 1, or an F-actin-binding fragment thereof.

TABLE 1
F-actin binding proteins that may be used in the conjugate of
the invention. Adapted from Table S2 of Giampazolias (2021).

| | Human | | Mouse | | | |
|---|---|---|---|---|---|---|
| | gene | Full human | gene | Chromosome | Genome | Full mouse |
| FABP | symbol | gene name | symbol | location | coordinates | gene name |
| 1 | ACTR2 | Actin-related | Actr2 | Chr11 | Chr11: 20062304- | ARP2 actin- |
| | | protein 2 | | 12.88 cM | 20112913 (−) | related protein |
| | | | | | GRCm38 | 2 |
| 2 | ACTR3 | Actin-related | Actr3 | Chr1 54.7 cM | Chr1: 125392905- | ARP3 actin- |
| | | protein 3 | | | 125435727 (−) | related protein |
| | | | | | GRCm38 | 3 |
| 3 | ACTN1 | Alpha-Actinin | Actn1 | Chr12 | Chr12: 80167547- | Actinin, alpha 1 |
| | | | | 36.49 cM | 80260371 (−) | |
| | | | | | GRCm38 | |
| 4 | ANLN | Anillin | Anln | Chr9 8.93 cM | Chr9: 22332012- | Anillin, actin |
| | | | | | 22389188 (−) | binding protein |
| | | | | | GRCm38 | |
| 5 | CAPZA1 | Capping actin | Capza1 | Chr3 | Chr3: 104822781- | Capping protein |
| | | protein of | | 45.88 cM | 104864505 (−) | (actin filament) |
| | | muscle Z-line | | | GRCm38 | muscle Z-line, |
| | | subunit alpha 1 | | | | alpha 1 |
| 6 | CAPZA2 | Capping actin | Capza2 | Chr6 7.96 cM | Chr6: 17637010- | Capping protein |
| | | protein of | | | 17666972 (+) | (actin filament) |
| | | muscle Z-line | | | GRCm38 | muscle Z-line, |
| | | subunit alpha 2 | | | | alpha 2 |
| 7 | CAPZB | Capping actin | Capzb | Chr4 | Chr4: 139192899- | Capping protein |
| | | protein of | | 70.59 cM | 139291818 (+) | (actin filament) |
| | | muscle Z-line | | | GRCm38 | muscle Z-line, |
| | | subunit beta | | | | beta |
| 8 | CARMIL1 | Capping protein | Carmil1 | Chr13 | Chr13: 24012481- | Capping protein |
| | | regulator and | | 10.05 cM | 24280802 (−) | regulator and |
| | | myosin 1 linker | | | GRCm38 | myosin 1 linker |
| | | 1 | | | | 1 |
| 9 | CDC42 | Cell division | Cdc42 | Chr4 | Chr4: 137319696- | Cell division |
| | | control protein | | 69.83 cM | 137357720 (−) | cycle 42 |
| | | 42 homolog | | | GRCm38 | |
| 10 | CFL1 | Cofilin 1 | Cfl1 | Chr19 | Chr19: 5490455- | Cofilin 1, non- |
| | | | | 4.34 cM | 5494031 (+) | muscle |
| | | | | | GRCm38 | |
| 11 | CFL2 | Cofilin 2 | Cfl2 | Chr12 | Chr12: 54858815- | Cofilin 2, |
| | | | | 23.75 cM | 54862877 (−) | muscle |
| | | | | | GRCm38 | |
| 12 | EZR | Ezrin | Ezr | Chr17 | Chr17: 6738131- | Ezrin |
| | | | | 4.38 cM | 6782780 (−) | |
| | | | | | GRCm38 | |
| 13 | FSCN1 | Fascin | Fscn1 | Chr5 | Chr5: 142960355- | Fascin actin- |
| | | | | 81.84 cM | 142973189 (+) | bundling protein |
| | | | | | GRCm38 | 1 |
| 14 | FLNA | Filamin A | Flna | ChrX | ChrX: 74223461- | Filamin, alpha |
| | | | | 37.89 cM | 74249820 (−) | |
| | | | | | GRCm38 | |
| 15 | FLNB | Filamin B | Flnb | Chr14 | Chr14: 7817957- | Filamin, beta |
| | | | | 4.59 cM | 7951588 (+) | |
| | | | | | GRCm38 | |
| 16 | | Fimbrin | Pls1 | Chr9 | Chr9: 95752642- | Plastin 1 |
| | | | | 50.22 cM | 95845306 (−) | |
| | | | | | GRCm38 | |
| 17 | FMN1 | Formin-1 | Fmn1 | Chr2 57.3 cM | Chr2: 113327736- | Formin 1 |
| | | | | | 113716767 (+) | |
| | | | | | GRCm38 | |
| 18 | FMN2 | Formin-2 | Fmn2 | Chr1 | Chr1: 174501825- | Formin 2 |
| | | | | 81.04 cM | 174822729 (+) | |
| | | | | | GRCm38 | |
| 19 | GSN | Gelsolin | Gsn | Chr2 23.5 cM | Chr2: 35256380- | Gelsolin |
| | | | | | 35307892 (+) | |
| | | | | | GRCm38 | |
| 20 | CCDC88A | Girdin | Ccdc88a | Chr11 | Chr11: 29373658- | Coiled coil |
| | | | | 16.61 cM | 29510808 (+) | domain |
| | | | | | GRCm38 | containing 88A |
| 21 | CAPG | Macrophage- | Capg | Chr6 | Chr6: 72544391- | Capping protein |
| | | capping protein | | 32.27 cM | 72562983 (+) | (actin filament), |
| | | | | | GRCm38 | gelsolin-like |
| 22 | MSN | Moesin | Msn | ChrX | ChrX: 96096045- | Moesin |
| | | | | 42.65 cM | 96168553 (+) | |
| | | | | | GRCm38 | |
| 23 | MYH1 | Myosin heavy | Myh1 | Chr11 | Chr11: 67200102- | Myosin, heavy |
| | | chain 1 | | 40.59 cM | 67224577 (+) | polypeptide 1, |
| | | | | | GRCm38 | skeletal muscle, |
| | | | | | | adult |
| 24 | MYH10 | Myosin heavy | Myh10 | Chr11 | Chr11: 68691915- | Myosin, heavy |
| | | chain 10 | | 41.95 cM | 68816626 (+) | polypeptide 10, |
| | | | | | GRCm38 | non-muscle |
| 25 | MYH11 | Myosin heavy | Myh11 | Chr16 | Chr16: 14194535- | Myosin, heavy |
| | | chain 11 | | 9.71 cM | 14291372 (−) | polypeptide 11, |
| | | | | | GRCm38 | smooth muscle |
| 26 | MYH13 | Myosin heavy | Myh13 | Chr11 | Chr11: 67327103- | Myosin, heavy |
| | | chain 13 | | 40.85 cM | 67371587 (+) | polypeptide 13, |
| | | | | | GRCm38 | skeletal muscle |
| 27 | MYH14 | Myosin heavy | Myh14 | Chr7 | Chr7: 44605803- | Myosin, heavy |
| | | chain 14 | | 28.85 cM | 44670843 (−) | polypeptide 14 |
| | | | | | GRCm38 | |
| 28 | MYH15 | Myosin heavy | Myh15 | Chr16 | Chr16: 49057486- | Myosin, heavy |
| | | chain 15 | | 30.75 cM | 49199104 (+) | chain 15 |
| | | | | | GRCm38 | |
| 29 | MYH16 | Myosin heavy | | | | |
| | | chain 16 | | | | |
| 30 | MYH2 | Myosin heavy | Myh2 | Chr11 | Chr11: 67171027- | Myosin, heavy |
| | | chain 2 | | 40.59 cM | 67197517 (+) | polypeptide 2, |
| | | | | | GRCm38 | skeletal muscle, |
| | | | | | | adult |
| 31 | MYH3 | Myosin heavy | Myh3 | Chr11 | Chr11: 67078300- | Myosin, heavy |
| | | chain 3 | | 40.59 cM | 67102291 (+) | polypeptide 3, |
| | | | | | GRCm38 | skeletal muscle, |
| | | | | | | embryonic |
| 32 | MYH4 | Myosin heavy | Myh4 | Chr11 | Chr11: 67238029- | Myosin, heavy |
| | | chain 4 | | 40.59 cM | 67260446 (+) | polypeptide 4, |
| | | | | | GRCm38 | skeletal muscle |
| 33 | MYH6 | Myosin heavy | Myh6 | Chr14 | Chr14: 54941921- | Myosin, heavy |
| | | chain 6 | | 28.01 cM | 54966927 (−) | polypeptide 6, |
| | | | | | GRCm38 | cardiac muscle, |
| | | | | | | alpha |
| 34 | MYH7 | Myosin heavy | Myh7 | Chr14 | Chr14: 54970684- | Myosin, heavy |
| | | chain 7 | | 28.01 cM | 54994626 (−) | polypeptide 7, |
| | | | | | GRCm38 | cardiac muscle, |
| | | | | | | beta |
| 35 | MYH8 | Myosin heavy | Myh8 | Chr11 | Chr11: 67277124- | Myosin, heavy |
| | | chain 8 | | 40.59 cM | 67308634 (+) | polypeptide 8, |
| | | | | | GRCm38 | skeletal muscle, |
| | | | | | | perinatal |
| 36 | MYH9 | Myosin heavy | Myh9 | Chr15 | Chr15: 77760587- | Myosin, heavy |
| | | chain 9 | | 36.81 cM | 77842175 (−) | polypeptide 8, |
| | | | | | GRCm38 | non-muscle |
| 37 | MYO3A | Myosin IIIA | Myo3a | Chr2 | Chr2: 22227503- | Myosin IIIA |
| | | | | 15.15 cM | 22618252 (+) | |
| | | | | | GRCm38 | |
| 38 | MYO3B | Myosin IIIB | Myo3b | Chr2 | Chr2: 70039126- | Myosin IIIB |
| | | | | 41.05 cM | 70429195 (+) | |
| | | | | | GRCm38 | |
| 39 | MYO9A | Myosin IXA | Myo9a | Chr9 | Chr9: 59750896- | Myosin IXA |
| | | | | 32.13 cM | 59928866 (+) | |
| | | | | | GRCm38 | |
| 40 | MYO9B | Myosin IXB | Myo9b | Chr8 | Chr8: 71272714- | Myosin IXB |
| | | | | 34.43 cM | 71360713 (+) | |
| | | | | | GRCm38 | |
| 41 | MYL5 | Myosin light | | | | |
| | | chain 5 | | | | |
| 42 | MYL1 | Myosin light | Myl1 | Chr1 | Chr1: 66924295- | Myosin, light |
| | | chain 1 | | 33.71 cM | 66945404 (−) | polypeptide 1 |
| | | | | | GRCm38 | |
| 43 | MYL2 | Myosin light | Myl2 | Chr5 Syntenic | Chr5: 122100951- | Myosin, light |
| | | chain 2 | | | 122113472 (+) | polypeptide 2, |
| | | | | | GRCm38 | regulatory, |
| | | | | | | cardiac, slow |
| 44 | MYL3 | Myosin light | Myl3 | Chr9 | Chr9: 110763678- | Myosin, light |
| | | chain 3 | | 60.69 cM | 110769802 (+) | polypeptide 3 |
| | | | | | GRCm38 | |
| 45 | MYL4 | Myosin light | Myl4 | Chr11 | Chr11: 104546017- | Myosin, light |
| | | chain 4 | | 67.79 cM | 104587215 (+) | polypeptide 4 |
| | | | | | GRCm38 | |
| 46 | MYL6 | Myosin light | Myl6 | Chr10 | Chr10: 128490859- | Myosin, light |
| | | chain 6 | | 76.75 cM | 128493886 (−) | polypeptide 6, |
| | | | | | GRCm38 | alkali, smooth |
| | | | | | | muscle and |
| | | | | | | non-muscle |
| 47 | MYL6B | Myosin light | Myl6b | Chr10 | Chr10: 128494157- | Myosin, light |
| | | chain 6B | | 76.77 cM | 128498685 (−) | polypeptide 6B |
| | | | | | GRCm38 | |
| 48 | MYL7 | Myosin light | Myl7 | Chr11 | Chr11: 5896637- | Myosin, light |
| | | chain 7 | | 3.88 cM | 5898782 (−) | polypeptide 7, |
| | | | | | GRCm38 | regulatory |
| 49 | MYL9 | Myosin light | Myl9 | Chr2 | Chr2: 156775420- | Myosin, light |
| | | chain 9 | | 77.77 cM | 156781658 (+) | polypeptide 9, |
| | | | | | GRCm38 | regulatory |
| 50 | MYLK2 | Myosin light | Mylk2 | Chr2 | Chr2: 152911352- | Myosin, light |
| | | chain kinase 2, | | 75.41 cM | 152923068 (+) | polypeptide |
| | | skeletal/cardiac | | | GRCm38 | kinase 2, |
| | | muscle | | | | skeletal muscle |
| 51 | MYLK | Myosin light | Mylk | Chr16 | Chr16: 34745210- | Myosin, light |
| | | chain kinase, | | 24.49 cM | 35002420 (+) | polypeptide |
| | | smooth muscle | | | GRCm38 | kinase |
| 52 | MYLIP | Myosin | Mylip | Chr13 | Chr13: 45389742- | Myosin |
| | | regulatory light | | 21.88 cM | 45411940 (+) | regulatory light |
| | | chain | | | GRCm38 | chain |
| | | interacting | | | | interacting |
| | | protein | | | | protein |
| 53 | MYO5A | Myosin VA | Myo5a | Chr9 | Chr9: 75071015- | Myosin VA |
| | | | | 42.26 cM | 75223688 (+) | |
| | | | | | GRCm38 | |
| 54 | MYO5B | Myosin VB | Myo5b | Chr18 | Chr18: 74442364- | Myosin VB |
| | | | | 50.7 cM | 74772698 (+) | |
| | | | | | GRCm38 | |
| 55 | MYO5C | Myosin VC | Myo5c | Chr9 42.3 cM | Chr9: 75232020- | Myosin VC |
| | | | | | 75305451 (+) | |
| | | | | | GRCm38 | |
| 56 | MYO6 | Myosin VI | Myo6 | Chr9 | Chr9: 80165031- | Myosin VI |
| | | | | 43.98 cM | 80311729 (+) | |
| | | | | | GRCm38 | |
| 57 | MYO7A | Myosin VIIA | Myo7a | Chr7 | Chr7: 98051060- | Myosin VIIA |
| | | | | 53.57 cM | 98119524 (−) | |
| | | | | | GRCm38 | |
| 58 | MYO7B | Myosin VIIB | Myo7b | Chr18 | Chr18: 31959234- | Myosin VIIB |
| | | | | 17.89 cM | 32036931 (−) | |
| | | | | | GRCm38 | |
| 59 | MYO10 | Myosin X | Myo10 | Chr15 | Chr15: 25622550- | Myosin X |
| | | | | 9.36 cM | 25813673 (+) | |
| | | | | | GRCm38 | |
| 60 | MYO15A | Myosin XVA | Myo15 | Chr11 | Chr11: 60469339- | Myosin XV |
| | | | | 37.81 cM | 60528369 (+) | |
| | | | | | GRCm38 | |
| 61 | MYO18A | Myosin XVIIIA | Myo18a | Chr11 | Chr11: 77763246- | Myosin XVIIIA |
| | | | | 46.74 cM | 77865980 (+) | |
| | | | | | GRCm38 | |
| 62 | MYO18B | Myosin XVIIIB | Myo18b | Chr5 54.8 cM | Chr5: 112688876- | Myosin XVIIIB |
| | | | | | 112896362 (−) | |
| | | | | | GRCm38 | |
| 63 | MYO1A | Myosin-IA | Myo1a | Chr10 | Chr10: 127705170- | Myosin IA |
| | | | | 74.62 cM | 127720940 (+) | |
| | | | | | GRCm38 | |
| 64 | MYO1B | Myosin-IB | Myo1b | Chr1 | Chr1: 51749758- | Myosin IB |
| | | | | 26.58 cM | 51916063 (−) | |
| | | | | | GRCm38 | |
| 65 | MYO1C | Myosin-IC | Myo1c | Chr11 | Chr11: 75650504- | Myosin IC |
| | | | | 45.92 cM | 75673910 (+) | |
| | | | | | GRCm38 | |
| 66 | MYO1D | Myosin-ID | Myo1d | Chr11 | Chr11: 80482126- | Myosin ID |
| | | | | 47.95 cM | 80780025 (−) | |
| | | | | | GRCm38 | |
| 67 | MYO1E | Myosin-IE | Myo1e | Chr9 | Chr9: 70207350- | Myosin IE |
| | | | | 39.41 cM | 70399766 (+) | |
| | | | | | GRCm38 | |
| 68 | MYO1F | Myosin-IF | Myo1f | Chr17 | Chr17: 33555707- | Myosin IF |
| | | | | 17.98 cM | 33607764 (+) | |
| | | | | | GRCm38 | |
| 69 | MYO1G | Myosin-IG | Myo1g | Chr11 4.3 cM | Chr11: 6506548- | Myosin IG |
| | | | | | 6520960 (−) | |
| | | | | | GRCm38 | |
| 70 | MYO1H | Myosin-IH | Myo1h | Chr5 | Chr5: 114289058- | Myosin IH |
| | | | | 55.99 cM | 114364576 (+) | |
| | | | | | GRCm38 | |
| 71 | MYLL1 | Myosin, light | | | | |
| | | chain, cardiac | | | | |
| | | muscle-Like 1 | | | | |
| 72 | DIAPH1 | Protein | Diaph1 | Chr18 | Chr18: 37843601- | Diaphanous |
| | | diaphanous | | 19.71 cM | 37935476 (−) | related formin 1 |
| | | homolog 1 | | | GRCm38 | |
| 73 | RDX | Radixin | Rdx | Chr9 | Chr9: 52047150- | Radixin |
| | | | | 28.62 cM | 52088738 (+) | |
| | | | | | GRCm38 | |
| 74 | SCIN | Scinderin | Scin | Chr12 | Chr12: 40059769- | Scinderin |
| | | | | 18.06 cM | 40134228 (−) | |
| | | | | | GRCm38 | |
| 75 | SPTA1 | Spectrin alpha, | Spta1 | Chr1 | Chr1: 174172776- | Spectrin alpha, |
| | | erythrocytic 1 | | 80.97 cM | 174248450 (+) | erythrocytic 1 |
| | | | | | GRCm38 | |
| 76 | SPTAN1 | Spectrin alpha, | Sptan1 | Chr2 | Chr2: 29965560- | Spectrin alpha, |
| | | nonerythrocytic | | 20.93 cM | 30031451 (+) | nonerythrocytic |
| | | 1 | | | GRCm38 | 1 |
| 77 | SPTB | Spectrin beta, | Sptb | Chr12 | Chr12: 76580488- | Spectrin beta, |
| | | erythrocytic | | 33.73 cM | 76710547 (−) | erythrocytic |
| | | | | | GRCm38 | |
| 78 | SPTBN1 | Spectrin beta, | Sptbn1 | Chr11 | Chr11: 30099395- | Spectrin beta, |
| | | nonerythrocytic | | 17.44 CM | 30268175 (−) | nonerythrocytic |
| | | 1 | | | GRCm38 | 1 |
| 79 | SPTBN2 | Spectrin beta, | Sptbn2 | Chr19 4.1 cM | Chr19: 4711167- | Spectrin beta, |
| | | nonerythrocytic | | | 4752360 (+) | nonerythrocytic |
| | | 2 | | | GRCm38 | 2 |
| 80 | SPTBN4 | Spectrin beta, | Sptbn4 | Chr7 | Chr7: 27356383- | Spectrin beta, |
| | | nonerythrocytic | | 15.88 cM | 27447686 (−) | nonerythrocytic |
| | | 4 | | | GRCm38 | 4 |
| 81 | SPIRE1 | Spire type actin | Spire1 | Chr18 | Chr18: 67488209- | Spire type actin |
| | | nucleation | | 39.99 cM | 67610790 (−) | nucleation |
| | | factor 1 | | | GRCm38 | factor 1 |
| 82 | SPIRE2 | Spire type actin | Spire2 | Chr8 72.1 cM | Chr8: 123332713- | Spire type actin |
| | | nucleation | | | 123369515 (+) | nucleation |
| | | factor 2 | | | GRCm38 | factor 2 |
| 83 | TMOD1 | Tropomodulin-1 | Tmod1 | Chr4 | Chr4: 46038940- | Tropomodulin 1 |
| | | | | 24.49 cM | 46116032 (+) | |
| | | | | | GRCm38 | |
| 84 | TMOD2 | Tropomodulin-2 | Tmod2 | Chr9 | Chr9: 75565621- | Tropomodulin 2 |
| | | | | 42.31 cM | 75611325 (−) | |
| | | | | | GRCm38 | |
| 85 | TMOD3 | Tropomodulin-3 | Tmod3 | Chr9 42.3 cM | Chr9: 75497784- | Tropomodulin 3 |
| | | | | | 75559657 (−) | |
| | | | | | GRCm38 | |
| 86 | TPM1 | Tropomyosin | Tpm1 | Chr9 | Chr9: 67022590- | Tropomyosin 1, |
| | | alpha-1 chain | | 36.27 cM | 67049406 (−) | alpha |
| | | | | | GRCm38 | |
| 87 | TPM3 | Tropomyosin | Tpm3 | Chr3 39.2 cM | Chr3: 90072651- | Tropomyosin 3, |
| | | alpha-3 Chain | | | 90100902 (+) | gamma |
| | | | | | GRCm38 | |
| 88 | TPM4 | Tropomyosin | Tpm4 | Chr8 | Chr8: 72135288- | Tropomyosin 4 |
| | | alpha-4 chain | | 34.81 cM | 72153142 (+) | |
| | | | | | GRCm38 | |
| 89 | TPM2 | Tropomyosin | Tpm2 | Chr4 | Chr4: 43514711- | Tropomyosin 2, |
| | | beta chain | | 23.05 cM | 43523765 (−) | beta |
| | | | | | GRCm38 | |
| 90 | VASP | Vasodilator- | Vasp | Chr7 9.51 cM | Chr7: 19256929- | Vasodilator- |
| | | stimulated | | | 19271817 (−) | stimulated |
| | | phosphoprotein | | | GRCm38 | phosphoprotein |
| 91 | VIL1 | Villin1 | Vil1 | Chr1 | Chr1: 74409384- | Villin 1 |
| | | | | 38.54 cM | 74435560 (+) | |
| | | | | | GRCm38 | |

### Anti-F-Actin Antibodies

In embodiments where the conjugate is secreted, or in aspects where the vaccine or agent comprises a (protein) conjugate cytoskeleton-binding agent may be an antibody. The cytoskeleton-binding agent may be an antibody with a target-binding fragment specific for actin. The target-binding fragment may be specific for F-actin. The target-binding fragment may bind to both F-actin and G-actin. The antibody may be a target-binding fragment of an antibody (for example a Fab fragment) or a synthetic antibody fragment (for example a single chain Fv fragment [ScFv] or single-domain antibody/nanobody). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 792-799).

Monoclonal antibodies (mAbs) are useful in the methods of the invention and are a homogenous population of antibodies specifically targeting a single epitope on an antigen. Suitable monoclonal antibodies can be prepared using methods well known in the art (e.g. see Kohler, G.; Milstein, C. (1975). “Continuous cultures of fused cells secreting antibody of predefined specificity”. Nature 256 (5517): 495; Siegel DL (2002). “Recombinant monoclonal antibody technology”. Schmitz U, Versmold A, Kaufmann P, Frank HG (2000); “Phage display: a molecular tool for the generation of antibodies—a review”. Placenta. 21 Suppl A: S106-12. Helen E. Chadd and Steven M. Chamow; “Therapeutic antibody expression technology,” Current Opinion in Biotechnology 12, no. 2 (Apr. 1, 2001): 188-194; McCafferty, J.; Griffiths, A.; Winter, G.; Chiswell, D. (1990). “Phage antibodies: filamentous phage displaying antibody variable domains”. Nature 348 (6301): 552-554; “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 792-799)).

Polyclonal antibodies are useful in the methods of the invention. Monospecific polyclonal antibodies are preferred. Suitable polyclonal antibodies can be prepared using methods well known in the art.

Fragments of antibodies, such as Fab and Fab2 fragments may also be used as can genetically engineered antibodies and antibody fragments. The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the VH and VL partner domains are inked via a flexible oligopeptide (Bird et al (1988) Science 242, 423; Huston et al (1988) Proc. Natl. Acad. Sd. USA 85, 5879) and single domain antibodies (sdAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the VH and VL partner domains are covalently inked, e.g. directly, by a peptide or by a flexible oligopeptide. Fab, Fv, ScFv and sdAb antibody fragments can all be expressed in and secreted from E. coli, thus allowing the facile production of large amounts of the said fragments.

Whole antibodies, and F(ab′)2 fragments are “bivalent”. By “bivalent” we mean that the said antibodies and F(ab′)2 fragments have two antigen combining sites. In contrast, Fab, Fv, ScFv and sdAb fragments are monovalent, having only one antigen combining site. Synthetic antibodies which bind to a target discussed herein may also be made using phage display technology as is well known in the art (e.g. see “Phage display: a molecular tool for the generation of antibodies—a review”. Placenta. 21 Suppl A: S106-12. Helen E. Chadd and Steven M. Chamow; “Phage antibodies: filamentous phage displaying antibody variable domains”. Nature 348 (6301): 552-554).

### AFFIMER®s

The cytoskeleton-binding agent may be an AFFIMER®. AFFIMER®s are small binding proteins which mimic antibodies in terms of molecular recognition characteristics (Woodman, 2005; Tiede, 2014). They exhibit higher stability in comparison to antibodies. They are recombinant proteins which can be engineered to bind a target of interest. All AFFIMER®s consist of an alpha-helix on top of an anti-parallel beta-sheet, as well as two peptide loops. These peptide loops can be randomised to bind to the desired target. The skilled person is able to select or design affimers directed to a desired target. The affimer may have affinity and/or avidity for both F-actin and G-actin. The affimer may be specific for F-actin. The affimer may be fused to the antigen, optionally via a linker, via the N- or C-terminus of the affimer.

### Professional/Non-Professional Antigen Presenting Cells

Certain immune cells, such as dendritic cells and particular macrophage populations, are considered “professional” antigen presenting cells (professional APCs). While most cell types can perform antigen presentation on MHC class I molecules (specifically when the antigen has been synthesised intracellularly (endogenous antigen)), professional APCs can additionally process and present exogenous antigens on MHC class II and/or cross-present exogenous antigens on MHC class I molecules. Whether a professional APC engages in antigen presentation on MHC-II or MHC-I is also affected by their cell type and the nature of the antigen. Importantly, cDC1, a type of DC, are particularly adept at XP of cell-associated antigens, such as tumour antigens, due in part to their unique receptor expression pattern and have critical roles in anti-tumour immunity.

### Adjuvant

The vaccine described herein may be administered in combination with at least one adjuvant. An adjuvant is a compound that is administered in combination with a vaccine that prolongs, enhances or improves an immune response. Various adjuvants that may be used include oil emulsions (e.g., incomplete Freund's adjuvants, complete Freund's adjuvants), mineral compounds (e.g. alum, tocopherol), bacterial products (e.g. Bordetella pertussis toxin), saponins or immune-stimulating complexes. The adjuvant may be selected from the group consisting of a) aluminum salts comprising aluminum hydroxide, aluminum phosphate, aluminum sulphate phosphate; b) inulin; c) algammulin which is a combination of inulin and aluminium hydroxide; d) monophosphoryl lipid A (MPL); e) resiquimod; f) muramyl dipeptide (MDP); g) N-glycolyl dipeptide (GMDP); h) polylC; i) CpG oligonucleotide; j) aluminum hydroxide with MPL; k) any water in oil emulsion; l) any oil in water emulsion that contains one or more of the following constituents: squalene or its analogues or any pharmaceutically acceptable oil, tween-80, sorbitantrioleate, alpha-tocopherol, cholecalciferol and aqueous buffer, or any of the analogues and derivatives of the molecules thereof wherein one or two or more combination of any of the aforementioned adjuvants when formulated with Zika virus and Japanese encephalitis virus antigens elicits immune response against the virus. In one preferred embodiment, the adjuvant is aluminum hydroxide with 0.25 mg to 1.0 mg of aluminum content per vaccine dose.

### Pharmaceutical Compositions

Pharmaceutical compositions may be prepared using a pharmaceutically acceptable “carrier” composed of materials that are considered safe and effective. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In some embodiments, this term refers to molecular entities and compositions approved by a regulatory agency of the US federal or a state government, as the GRAS list under section 204(s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans.

The term “carrier” refers to diluents, binders, lubricants and disintegrants. Those with skill in the art are familiar with such pharmaceutical carriers and methods of compounding pharmaceutical compositions using such carriers.

The pharmaceutical compositions provided herein may include one or more excipients, e.g., solvents, solubility enhancers, suspending agents, buffering agents, isotonicity agents, antioxidants or antimicrobial preservatives. When used, the excipients of the compositions will not adversely affect the stability, bioavailability, safety, and/or efficacy of the active ingredients, i.e. the vectors, cells and or chimeric receptors, used in the composition. Thus, the skilled person will appreciate that compositions are provided wherein there is no incompatibility between any of the components of the dosage form. Excipients may be selected from the group consisting of buffering agents, solubilizing agents, tonicity agents, chelating agents, antioxidants, antimicrobial agents, and preservatives.

### Routes of Administration

Medicaments and pharmaceutical compositions according to aspects of the present invention may be formulated for administration by a number of routes, including but not limited to, parenteral, intravenous, intra-arterial, intramuscular, intratumoural, oral and nasal. The medicaments and compositions may be formulated in fluid or solid form. Fluid formulations may be formulated for administration by injection to a selected region of the human or animal body.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

### Subject

The subject to be treated may be any animal or human. The subject is preferably mammalian, more preferably human. The subject may be a non-human mammal, but is more preferably human. The subject may be male or female. The subject may be a patient. Therapeutic uses may be in human or animals (veterinary use).

### Cancers

A “cancer” can comprise any one or more of the following: acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and Wilms' tumor. In some embodiments, the cancer is not a prostate cancer.

Cancers may be of a particular type. Examples of types of cancer include astrocytoma, carcinoma (e.g. adenocarcinoma, hepatocellular carcinoma, medullary carcinoma, papillary carcinoma, squamous cell carcinoma), glioma, lymphoma, medulloblastoma, melanoma, myeloma, meningioma, neuroblastoma, sarcoma (e.g. angiosarcoma, chrondrosarcoma, osteosarcoma).

Some cancers cause solid tumours. Such solid tumours may be located in any tissue, for example the pancreas, lung, breast, uterus, stomach, kidney or testis. In contrast, cancers of the blood, such as leukaemias, may not cause solid tumours—and may be referred to as liquid tumours.

The cancer that is the subject of the treatments and medical uses of the present invention may be selected from the lists provided above.

### Infectious Diseases

An infectious disease refers to any disease which may be transmitted between individuals or organisms. This includes a viral disease, bacterial disease, fungal disease or parasitic disease, transmitted by a virus, a bacterium, a fungus or a parasite, respectively. The infectious disease may be, for example, hepatitis, cytomegalovirus (CMV), sexually transmitted diseases (e.g. chlamydia or gonorrhoea), tuberculosis, HIV/acquired immune deficiency syndrome (AIDS), diphtheria, hepatitis B, hepatitis C, cholera, severe acute respiratory syndrome (SARS), the bird flu, COVID-19 and influenza.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Materials & Methods

### Mice

C57BL/6 mice were purchased from Charles River (Margate, UK). OT-I x Rag1^{-/-} and OT-II mice were bred at the animal facility of the Francis Crick Institute. Animal experiments were performed in accordance with national and institutional guidelines for animal care and were approved by the Francis Crick Institute Animal Ethics Committee and by the UK Home Office.

### Reagents

Rat-anti-DNGR-1 antibody (1F6) and isotype control antibody (MAC49) were provided by the Cell Services STP (Crick). Poly I:C was from Invivogen.

### Cells

HeLa cells stably transduced with LAOVA, muLAOVA, affimer 6-OVA or control affimer-OVA were grown in R10⁺, RPMI 1640 containing 10% FCS, 2 mM glutamine, 50 μM 2-mercaptoethanol, 100 units/mi penicillin, 100 μg/ml streptomycin, 2 mM glutamine, 1 mM pyruvate, 10 mM HEPES and 0.1 mM non-essential amino acids. The MutuDC1940 line was a kind gift from Hans Acha-Orbea and was cultured in IMDM medium containing 10% FCS and 50 μM 2-mercaptoethanol. All media and media supplements were from ThermoFisher Scientific except for FCS (Source Bioscience). Pre-activated OT-I or OT-II T cells were generated by culturing red blood cell-depleted spleen and lymph node cells from OT-I x Rag1 KO or OT-II mice at 1×10⁶ cells/mi in TC-flasks for 5 days in R10⁺ supplemented with SIINFEKL peptide (0.1 nM) and mIL-2 (100 U/ml) or OVA₃₂₉₋₃₃₉ (MHCII) peptide (1 μM) and mIL-2 (10 U/ml), respectively. Fresh mIL-2 was added on days 3 and 4 and the cultures were split 1:2 if necessary (usually on day 4).

### LAOVA Cloning

LAOVA, muLAOVA, affimer 6-OVA and control affimer-OVA fusion constructs with an 8×His tag at the C-terminal and appropriate overhang sequences for in-fusion cloning. Constructs were synthesised as gBlocks (Integrated DNA Technologies) and cloned into the sleeping beauty plasmid pSBbi-RP (Addgene). The plasmid was digested with SfiI and the gBlocks were ligated into the cut plasmid using standard in-fusion cloning reagents and protocol. The plasmids were transfected into HeLa cells using Lipofectamine2000 and stable lines were obtained after puromycin selection.

### In Vitro Cross-Presentation

Necrotic LAOVA or muLAOVA expressing HeLa cells, which had been exposed to ultraviolet (UV) irradiation (240 mJ/cm²) the day before, were added to Mutu DCs (1×10⁵/well) at the indicated ratio and cultured in 96-well round-bottom plates at 37° C. To facilitate dead cell uptake, plates were centrifuged at 1000 rpm for 3 min at the start of the incubation. Pre-activated OT-I or OT-II T cells (5×10⁴/well) were then added to 4 hr Mutu/dead cell co-cultures and T cell activation was determined by measuring IFN-γ in the supernatant of overnight cultures by ELISA.

In Vivo Immunization with Necrotic Cells

Mice were injected i.v. with 1×10⁶ UV-irradiated HeLa cells expressing LAOVA or muLAOVA, which had been mixed with poly I:C (25 μg/mouse) prior to injection, in PBS in a total volume of 0.2 ml. 6 days later red blood cell lysed splenocyte suspensions were prepared from spleens of injected mice and re-stimulated with SIINFEKL peptide (1 μM) over-night. Re-stimulated splenocyte cultures were stained with anti-CD8α, anti-CD44 and live/dead dye, fixed and stained with anti-IFN-γ antibody in permeabilization buffer.

### Intracellular Staining and Flow Cytometry

For staining of internal proteins, cells were fixed in paraformaldehyde (Fix & Perm kit, Nordic MUbio), blocked overnight in FACS buffer (PBS+1% FCS+2 mM EDTA) and stained with the indicated antibodies in permeabilization buffer (Fix & Perm kit, Nordic MUbio). Samples were acquired on a LSRFortessa (BD Biosciences). Data were analysed using FlowJo software (Treestar).

### Western Blot

Cell pellets were lysed by boiling in SDS-PAGE sample buffer and proteins were separated on a 4-20% Tris-glycine gradient gel (BioRad). Proteins were transferred onto a PVDF membrane using the trans blot turbo transfer system (BioRad). Membranes were blocked over-night in 5% milk (blocking buffer) and probed with the indicated antibodies in blocking buffer. Bands were visualized using an ImageQuant apparatus (Amersham) following brief soaking of the membrane in a solution containing a HRP chemiluminescent substrate (SuperSignal West Pico Plus, Thermo Scientific).

### Example 1—Targeting OVA to F-Actin Improves Retention of OVA Upon Cell Death

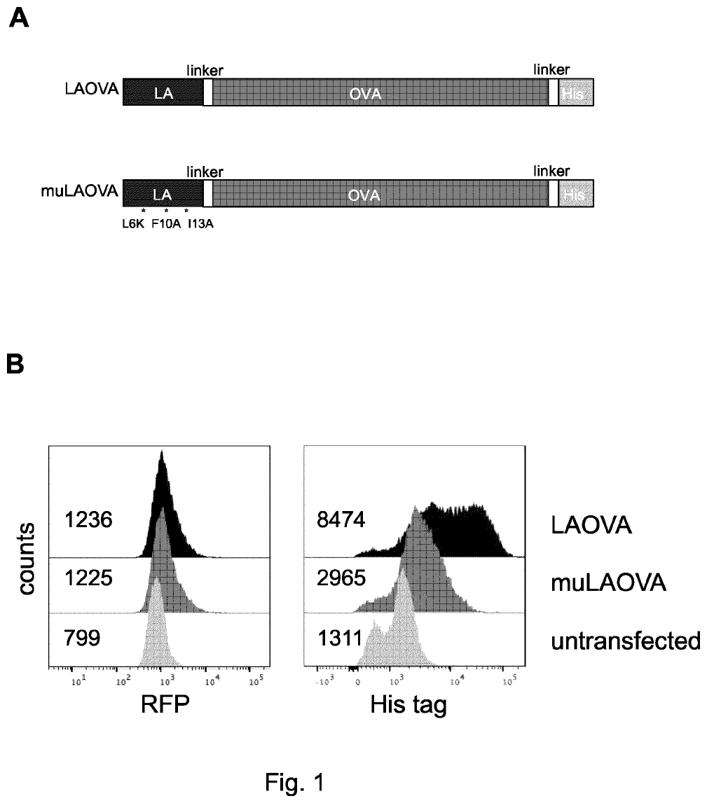

To investigate whether targeting an antigen to the actin cytoskeleton would improve T cell activation and potentiate an immune response, the inventors generated a test construct by fusing the sequence of the Lifeact (LA) peptide with that of ovalbumin (OVA). LA is a recombinant 17-amino acid peptide, derived from the actin-binding protein Abp140 from Saccharomyces cerevisiae (Riedl, 2008). OVA is an established model antigen and delta (Δ) OVA, which is a non-secretable version of OVA lacking the N-terminal signal peptide, was used in this study. The N-terminal LA sequence was connected to the ΔOVA sequence by a linker, and a second linker connected ΔOVA to an 8×His tag at the C-terminus (FIG. 1A). A control construct (muLAOVA) was also generated, which contained 3 point mutations in the LA peptide (L6K, F10A and 113A) (Belyy, 2020) that prevented the expressed fusion protein from binding to F-actin.

To determine the impact of targeting OVA to F-actin on the retention of the OVA antigen following induction of cell death, HeLa cells expressing either LAOVA or muLAOVA were subjected to UV treatment, and necrotic cell corpses were stained the next day with an anti-His-tag mAb. FIG. 1B shows that the staining of His-tagged fusion protein was disproportionately higher in cells expressing LAOVA compared to muLAOVA expressing cells, while the signal of (cytoplasmic) red fluorescent protein (RFP) was equally low in both cell lines. This indicates that, in contrast to cytoplasmic proteins, the release of proteins tethered to F-actin is attenuated following loss of membrane integrity. Therefore, this proof-of-concept experiment successfully demonstrated that targeting an antigen to F-actin improves retention of that antigen in necrotic cells.

### Example 2—Targeting OVA to F-Actin Leads to Cross-Presentation of OVA in a DNGR-1-Dependent Manner

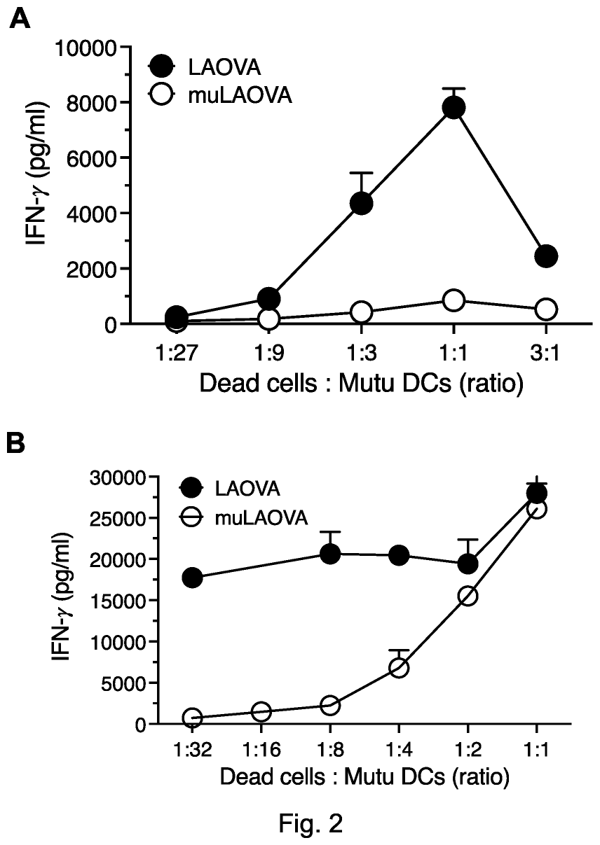

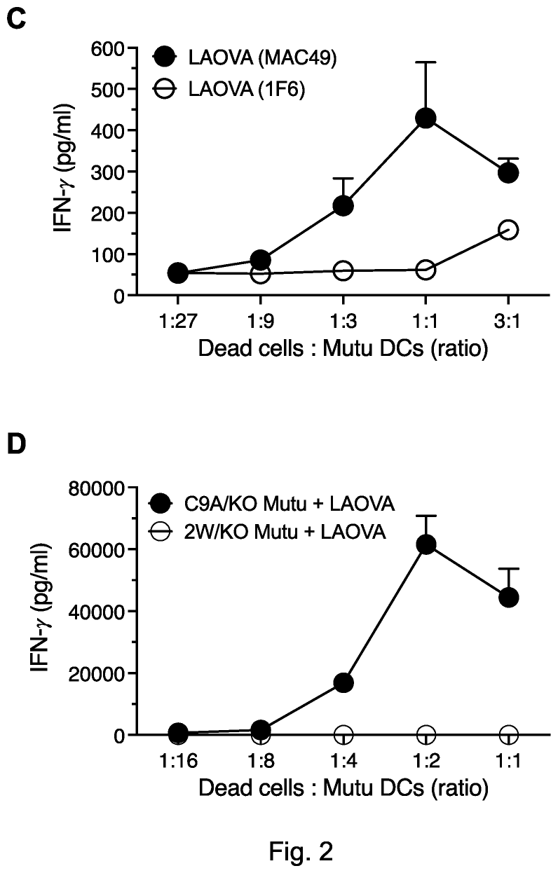

The effect of targeting OVA to F-actin on cross-presentation of OVA was then investigated. HeLa cells were transduced with either LAOVA or muLAOVA and irradiated with UV to induce necrosis. The necrotic HeLa cells were mixed with MutuDC cells, which consist of transformed mouse spleen cDC1s which naturally express DNGR-1. The ability of the MutuDC cells to cross-present dead cell-associated OVA was evaluated by measuring the stimulation of OT-I T cells (FIG. 2A) or OT-II T cells (FIG. 2B). As can be seen, the LAOVA conjugate led to efficient cross-presentation compared to the muLAOVA conjugate.

To further investigate the mechanism of this cross-presentation, the experiment was repeated in the presence of an anti-DNGR-1 antibody (1F6) or an isotype control antibody (MAC49) (as shown in FIG. 2C), and in the presence of either wildtype DNGR-1 (C9A/KO) or a double tryptophan mutant DNGR-1 (2W/KO) (as shown in FIG. 2D). The data shows that blocking DNGR-1 using either an anti-DNGR-1 antibody (FIG. 2C) or by genetic mutation (FIG. 2D) abrogated cross-presentation and downstream stimulation of OT-I cells. Therefore, cross-presentation of dead cell-associated OVA is dependent on DNGR-1.

### Example 3—Targeting OVA to F-Actin Promotes Cross-Priming of Endogenous OVA-Specific CD8⁻ T Cells In Vivo

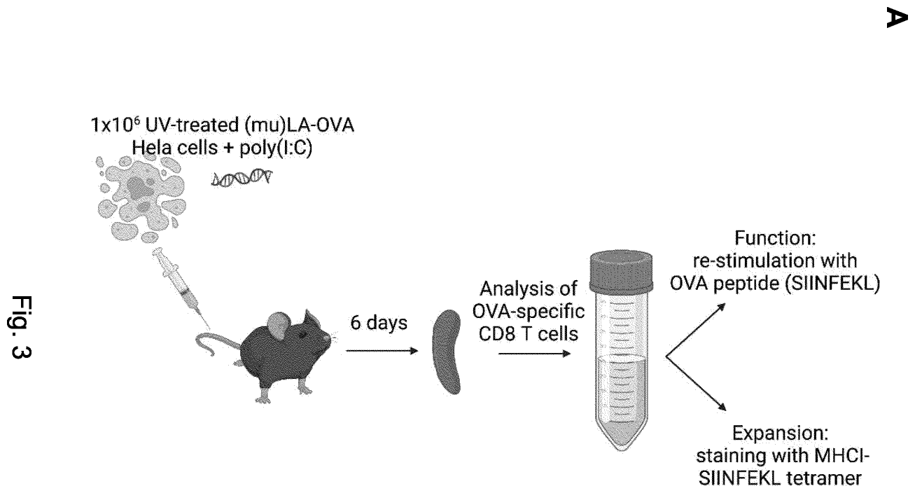

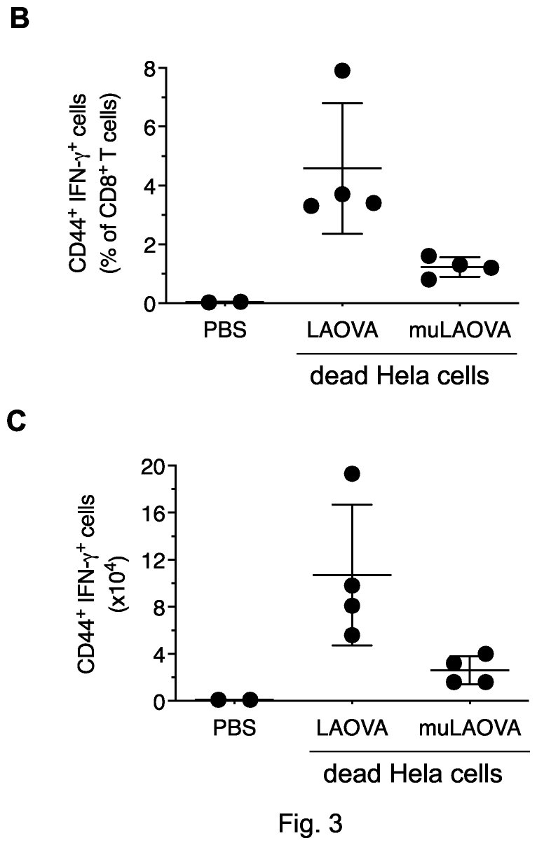

After establishing that OVA targeted to F-actin led to cross-presentation of OVA in vitro, the inventors sought to determine whether the effect is maintained in vivo. To this end, C57BL/6 mice were injected with UV-irradiated HeLa cells expressing either LAOVA or muLAOVA, mixed with poly I:C (25 μg/mouse) (as shown in schematic of FIG. 3A). OVA-specific CD8 T cell responses were evaluated by measuring the percentage of IFN-γ⁺ CD44⁺ double-positive cells of total CD8 T cells (FIG. 3B) and the absolute number of double positive cells (FIG. 3C). FIG. 3 highlights the increase in double positive cells following injection of LAOVA compared to muLAOVA.

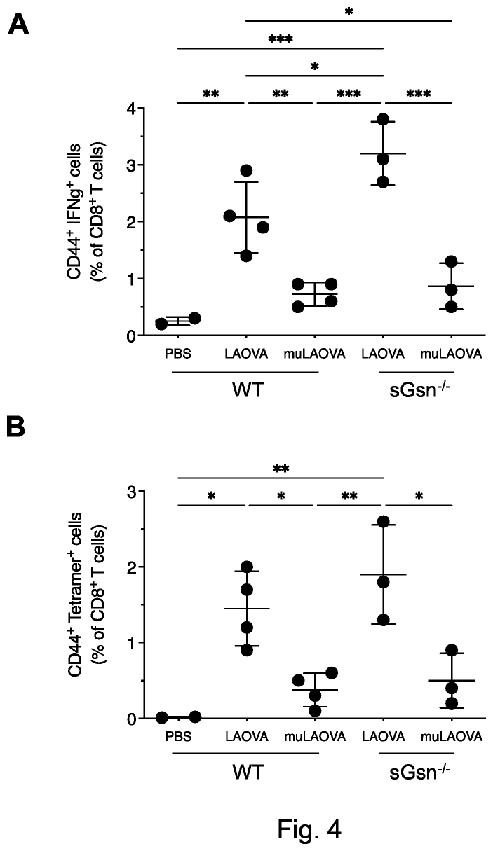

To further investigate the role of DNGR-1 in the cross-presentation of OVA in vivo, the experiment was repeated in secreted gelsolin-deficient (sGSN^{-/-}) mice. sGSN is the only known actin-binding protein (ABP) so far that competes with DNGR-1 for binding to F-actin filaments and therefore has a negative effect on DNGR-1 function. FIGS. 4A and 4B show that removing the inhibitory effect of sGSN leads to a further increase in cross-presentation of OVA in vivo.

### Example 4—Equal Expression of Affimer-OVA Fusion Proteins in HeLa Cells

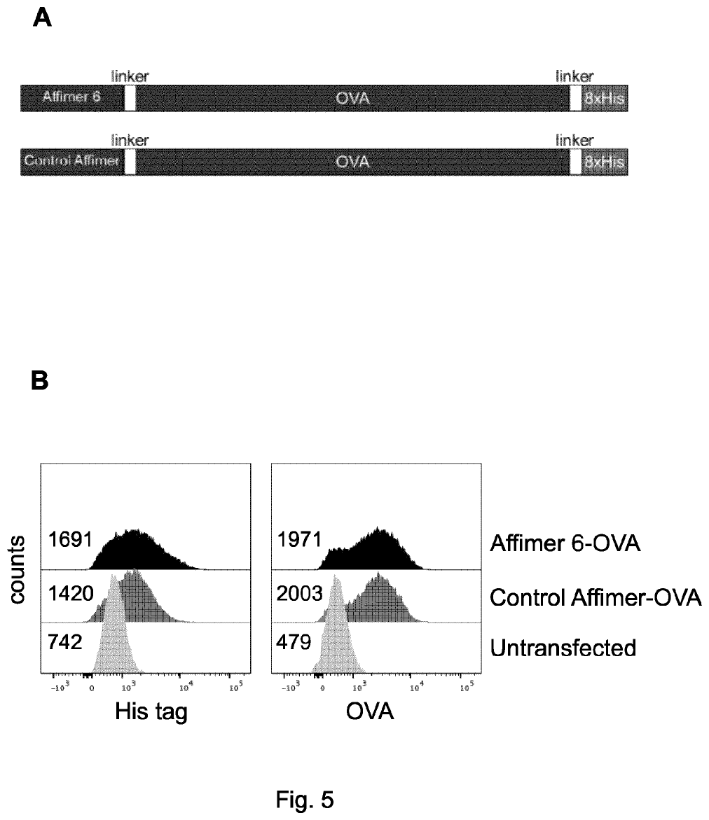

To determine if the beneficial effect of targeting OVA for cross-presentation by fusion to LA is maintained using other actin-binding agents, an F-actin specific affimer was fused to OVA. For these experiments, the affimer described as Affimer 6 in Tiede (2014) was used. This affimer has been shown to bind specifically to actin filaments (Lopata, 2018; Sugizaki, 2021). The amino acid sequence of Affimer 6 is shown in SEQ ID NO: 2. The N-terminal Affimer 6 sequence was connected to the ΔOVA sequence by a linker, and a second linker connected ΔOVA to an 8×His tag at the C-terminus (Figure SA). A control construct was also generated, which contained a control affimer that did not bind to F-actin. Any affimer that does not bind to F-actin may be used as the control affimer. FIG. 5B confirms that the amount of His-tagged OVA fusion protein is comparable in HeLa cells expressing either of these constructs.

### Example 5—Targeting OVA to F-Actin Using an Affimer is an Alternative Strategy to Improve Cross-Presentation of Dead Cell-Associated OVA

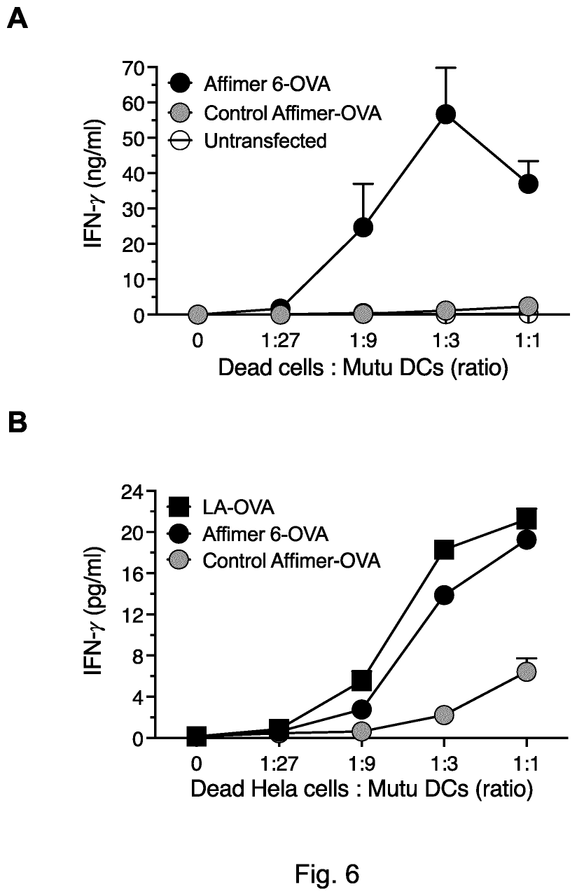

The experiment outlined in Example 2 was repeated using Affimer 6-OVA and Control Affimer-OVA to determine whether an affimer was able to maintain the improved cross-presentation of OVA seen using LA-OVA. FIG. 6A shows that irradiated cells expressing Affimer 6-OVA targeted to F-actin are cross-presented more efficiently compared to Control Affimer-OVA cells. Moreover, FIG. 6B shows that Affimer 6-OVA cells are cross-presented as efficiently as LA-OVA cells. As such, targeting an antigen to F-actin via an affimer is a viable strategy.

### SUMMARY

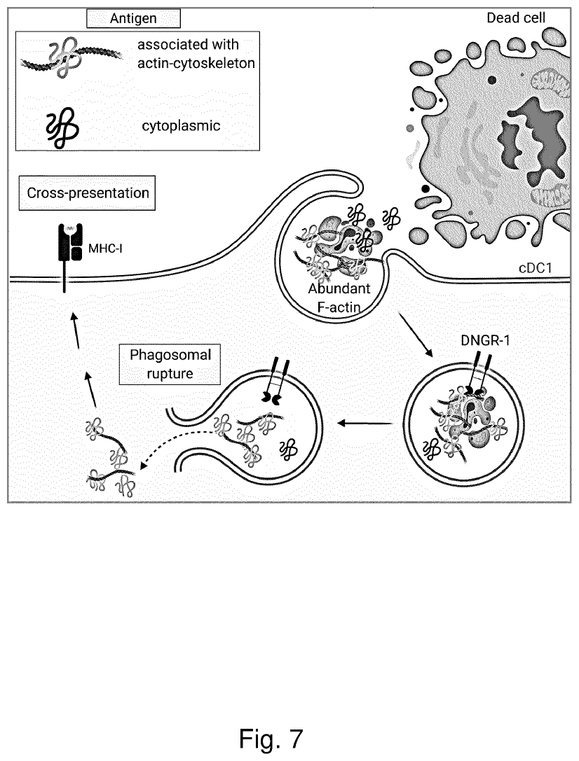

The preliminary results presented herein provide confirmation that conjugating an antigen to a cytoskeleton binding agent promotes priming of the immune system against the antigen. This can be achieved through fusion of the antigen to a peptide derived from an actin-binding protein or to an F-actin-specific affimer. In both of these examples, said priming is dependent on DNGR-1. FIG. 7 provides a summary of one embodiment of the invention. Following necrosis or other cell permeabilisation, antigens that remain associated with the actin-cytoskeleton are more likely to be internalised by an APC, such as cDC1. Cytosolic antigens are less likely to be internalised. DNGR-1 binding to F-actin triggers phagosomal rupture, releasing the antigen-F-actin complex into the cytoplasm of the APC, where the antigen may enter the MHC-I cross-presentation pathway.

### REFERENCES

A number of publications are cited above in order to more fully describe and disclose the invention and the state of the art to which the invention pertains. Full citations for these references are provided below. The entirety of each of these references is incorporated herein.
Ahrens, S. et al. F-actin is an evolutionarily conserved damage-associated molecular pattern recognized by DNGR-1, a receptor for dead cells. Immunity 36, 635-645 (2012)
Alloatti, A., et al., Critical role for Sec22b-dependent antigen crosspresentation in antitumor immunity. Journal of Experimental Medicine, 2017.214(8): p. 2231-2241.
Belyy, A., Merino, F., Sitsel, O., Raunser, S. (2020). Structure of the Lifeact-F-actin complex. PLoS Biol 18(11): e3000925. https://doi.org/10.1371/journal.pbio.3000925
Canton, J., et al., The receptor DNGR-1 signals for phagosomal rupture to promote cross-presentation of dead-cell-associated antigens. Nat Immunol, 2021. 22(2): p. 140-153.
Chang, J. (2021). Adenovirus Vectors: Excellent Tools for Vaccine Development. Immune Network, 21(1). https://doi.org/10.4110/in.2021.21.e6
Collin, M., & Bigley, V. (2018). Human dendritic cell subsets: an update. Immunology, 154(1), 3-20. https://doi.org/10.1111Amm.12888
Galluzzi, L., et al., Immunogenic cell death in cancer and infectious disease. Nature Reviews Immunology, 2017. 17(2): p. 97-111.
Galluzzi, L. et al. Molecular mechanisms of cell death: recommendations of the Nomenclature Committee on Cell Death 2018. Cell Death & Differentiation, 2018. 25: p. 486-541
Giampazolias, E. et al. Secreted gelsolin inhibits DNGR-1-dependent cross-presentation and cancer immunity. Cell (2021) 184: p. 4016-4031
Hanč, P., et al., Structure of the Complex of F-Actin and DNGR-1, a C-Type Lectin Receptor Involved in Dendritic Cell Cross-Presentation of Dead Cell-Associated Antigens. Immunity, 2015. 42(5): p. 839-849.
Huysamen, C., et al., CLEC9A is a novel activation C-type lectin-like receptor expressed on BDCA3+ dendritic cells and a subset of monocytes. Journal of Biological Chemistry, 2008. 283(24): p. 16693-16701.
Liang Y., et al. Development and Delivery Systems of mRNA Vaccines. Front. Bioeng. Biotechnol., 27 Jul. 2021
Lopata A., et al. Affimer proteins for F-actin: novel affinity reagents that label F-actin in live and fixed cells. Sci Rep. 2018 Apr. 26; 8(1):6572. doi: 10.1038/s41598-018-24953-4. PMID: 29700342; PMCID: PMC5920084.
Marichal T., et al., DNA released from dying host cells mediates aluminum adjuvant activity. Nat Med. 2011 Jul. 17; 17(8):996-1002. doi: 10.1038/nm.2403.
Minute L, Teijeira A, Sanchez-Paulete A R, et al. Cellular cytotoxicity is a form of immunogenic cell death Journal for ImmunoTherapy of Cancer 2020; 8:e000325. doi: 10.1136/jitc-2019-000325
Morse, M., Gwin, W., & Mitchell, D. (2021). Vaccine Therapies for Cancer: Then and Now. Targeted Oncology, 16(2), 121-152. https-/doi.org/10.1007/s11523-020-00788-w
Mylvaganam, S., Freeman, S., & Grinstein, S. (2021). The cytoskeleton in phagocytosis and macropinocytosis. Current Biology, 31(10), R619-R632. https-/doi.org/10.1016/j.cub.2021.01.036
Poulin, L. F., et al., DNGR-1 is a specific and universal marker of mouse and human Batf3-dependent dendritic cells in lymphoid and nonlymphoid tissues. Blood, 2012. 119(25): p. 6052-6062.
Riedl, J. et al. Lifeact: a versatile marker to visualize F-actin. Nature Methods, 2008. 5(7): p. 605-7.
Sancho, D., et al., Tumor therapy in mice via antigen targeting to a novel, DC-restricted C-type lectin. Journal of Clinical Investigation, 2008. 118(6): p. 2098-2110.
Sancho, D. et al. Identification of a dendritic cell receptor that couples sensing of necrosis to immunity. Nature 458, 899-903 (2009)
Salmon, H., et al., Expansion and Activation of CD103+ Dendritic Cell Progenitors at the Tumor Site Enhances Tumor Responses to Therapeutic PD-L1 and BRAF Inhibition. Immunity, 2016. 44(4): p. 924-938
Schlake, T., Thess, A., Fotin-Mleczek, M., & Kallen, K. (2012). Developing mRNA-vaccine technologies. RNA Biology, 9(11), 1319-1330. https://doi.org/10.4161/ma.22269
Sugizaki A., et al. POLArIS, a versatile probe for molecular orientation, revealed actin filaments associated with microtubule asters in early embryos. Proc Natl Acad Sci USA. 2021 Mar. 16; 118(11):e2019071118. doi: 10.1073/pnas.2019071118. PMID: 33674463; PMCID: PMC7980422.
Tang, R., et al. Ferroptosis, necroptosis, and pyroptosis in anticancer immunity. Journal of Hematology & Oncology volume 13, Article number: 110 (2020)
Theisen, D. J., et al., WDFY4 is required for cross-presentation in response to viral and tumor antigens. Science, 2018. 362(6415): p. 694-699
Tiede, C., et al., Adhiron: a stable and versatile peptide display scaffold for molecular recognition applications. Protein Engineering, Design and Selection, 2014. 27(5): p. 145-155, https://doi.org/10.1093/protein/gzu007
Wculek, S. K., et al., Dendritic cells in cancer immunology and immunotherapy. Nature Reviews Immunology, 2020. 20(1): p. 7-24.
Woodman, R. et al., Design and validation of a neutral protein scaffold for the presentation of peptide aptamers. J. Mol. Biol. 2005 Oct. 7; 352(5):1118-33. doi: 10.1016/j.jmb.2005.08.001
WO 2009/013484A1
WO 2013/088136
Zhang, J.-G. et al. The dendritic cell receptor Clec9A binds damaged cells via exposed actin filaments. Immunity 36, 646-657 (2012).

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A vaccine comprising a nucleic acid that encodes a conjugate comprising an antigen and an F-actin-binding moiety.

2. The vaccine according to claim 1, wherein the nucleic acid is mRNA.

3. The vaccine according to claim 1, wherein the nucleic acid is DNA.

4. The vaccine according to any one of claims 1 to 3, wherein the vaccine is a non-viral vaccine.

5. The vaccine according to any one of claims 1 to 3, wherein the vaccine is a viral vaccine.

6. The vaccine according to any preceding claim, wherein the F-actin-binding moiety is LifeAct, actinin, anilin, ezrin, fascin, filamin, F-tractin, an aptamer, an affimer, or an F-actin binding protein selected from Table 1, or a functional fragment thereof.

7. The vaccine according to any preceding claim, wherein the antigen is an endogenous antigen.

8. The vaccine according to claim 7, wherein the endogenous antigen comprises a tumour-associated antigen (TAA) or a tumour-specific antigen (TSA).

9. The vaccine according to any one of claims 1 to 6, wherein the antigen is an exogenous antigen.

10. The vaccine according to claim 9, wherein the exogenous antigen comprises a viral, bacterial or fungal antigen.

11. The vaccine according to any preceding claim, wherein the antigen is linked to the F-actin-binding moiety by a linker.

12. The vaccine according to any preceding claim, wherein the conjugate does not comprise a fluorescent protein.

13. The vaccine according to any preceding claim, wherein the vaccine comprises an adjuvant.

14. The vaccine according to claim 13, wherein the adjuvant is Alum.

15. The vaccine according to any preceding claim, wherein the vaccine comprises a pharmaceutically acceptable carrier.

16. The vaccine according to any preceding claim, wherein the vaccine induces a T cell response to the antigen in a subject following administration of the vaccine to the subject.

17. The vaccine according to any preceding claim, wherein the vaccine induces a B cell response to the antigen in a subject following administration of the vaccine to the subject.

18. The vaccine according to claim 16 or claim 17, wherein the subject has cancer, and the vaccine is an anti-cancer vaccine.

19. The vaccine according to claim 16 or claim 17, wherein the subject has an infectious disease, and the vaccine is an infectious disease vaccine.

20. The vaccine according to any preceding claim, wherein the vaccine is administered in combination with another therapeutic agent.

21. The vaccine according to any preceding claim, wherein the vaccine is a therapeutic vaccine.

22. The vaccine according to any one of claims 1 to 20, wherein the vaccine is a preventative vaccine.

23. The vaccine according to any preceding claim for use in a method of eliciting an immune response in a subject in need thereof.

24. An agent for use in a method of eliciting an immune response in a subject in need thereof, wherein the agent comprises a nucleic acid that encodes a conjugate comprising an antigen and an F-actin-binding moiety, and wherein the conjugate does not comprise a fluorescent protein.

25. The agent for use according to claim 23 or 24, wherein the nucleic acid is mRNA.

26. The agent for use according to claim 23 or 24, wherein the nucleic acid is DNA.

27. The agent for use according to any of claims 24 to 26, wherein the F-actin-binding moiety is LifeAct, actinin, anilin, ezrin, fascin, filamin, F-tractin, an aptamer, an affimer, or an F-actin binding protein selected from Table 1, or a functional fragment thereof.

28. The agent for use according to any one of claims 24 to 27, wherein the antigen is an endogenous antigen.

29. The agent for use according to claim 28, wherein the endogenous antigen comprises a tumour-associated antigen (TAA) or a tumour-specific antigen (TSA).

30. The agent for use according to any one of claims 24 to 27, wherein the antigen is an exogenous antigen.

31. The agent for use according to claim 30, wherein the exogenous antigen comprises a viral, bacterial or fungal antigen.

32. The agent for use according to any one of claims 24 to 31, wherein the antigen is inked to the F-actin-binding moiety by a linker.

33. The vaccine or agent for use according to any one of claims 23 to 32, wherein the immune response comprises a T cel response to the antigen.

34. The vaccine or agent for use according to any one of claims 23 to 32, wherein the immune response comprises a B cel response to the antigen.

35. The vaccine or agent for use according to any one of claims 23 to 34, wherein the subject is a cancer patient or is at risk of developing a cancer and wherein the immune response comprises an anti-cancer immune response.

36. The vaccine or agent for use according to any of claims 23 to 34, wherein the subject has an infectious disease and wherein the immune response comprises an immune response to the infectious disease.

37. A method of priming a lymphocyte to attack a target cell that expresses an antigen in a subject, the method comprising delivering an agent to a receiver cel,
wherein the agent comprises a nucleic acid that expresses a conjugate comprising the antigen and an F-actin-binding moiety in the receiver cell;
wherein the conjugate binds to F-actin that is exteriorised following receiver cell death;
and wherein the conjugate is then recognised by an antigen presenting cell (APC) which subsequently presents the antigen to the lymphocyte, thus priming the lymphocyte to recognise the antigen on the target cell.

38. An agent for use in a method of priming a lymphocyte to attack a target cell that expresses an antigen in a subject, the method comprising delivering the agent to a receiver cell,
wherein the agent comprises a nucleic acid that expresses a conjugate comprising the antigen and an F-actin-binding moiety in the receiver cell;
wherein the conjugate binds to F-actin that is exteriorised following receiver cell death;
and wherein the conjugate is then recognised by an antigen presenting cell (APC) which subsequently presents the antigen to the lymphocyte, thus priming the lymphocyte to recognise the antigen on the target cell.

39. The method according to claim 37 or the agent for the use according to claim 38, wherein the APC is a dendritic cel.

40. The method or the agent for use according to claim 39, wherein the dendritic cell is a type I conventional dendritic cell (cDC1).

41. The method or the agent for use according to any one of claims 37 to 40, wherein the lymphocyte is a T cell.

42. The method or the agent for use according to any one of claims 37 to 41, wherein the APC cross-presents the antigen on an MHC class I molecule.

43. The method or the agent for use according to claim 42, wherein the T cel is a CD8+ T cell.

44. The method or the agent for use according to any one of claims 37 to 41, wherein the antigen is presented on an MHC class II molecule.

45. The method or the agent for use according to claim 44, wherein the T eel is a CD4+ T cell.

46. The method or the agent for use according to any one of claims 37 to 40, wherein the lymphocyte is a B cel.

47. The method or the agent for use according to any of claims 37 to 46, wherein the nucleic acid is mRNA.

48. The method or the agent for use according to any of claims 37 to 46, wherein the nucleic acid is DNA.

49. The method or the agent for use according to any of claims 37 to 48, wherein the F-actin-binding moiety is LifeAct, actinin, anilin, ezrin, fascin, filamin, F-tractin, an aptamer, an affimer, or an F-actin binding protein selected from Table 1, or a functional fragment thereof.

50. The method or the agent for use according to any of claims 37 to 49, wherein the antigen is an endogenous antigen.

51. The method or the agent for use according to claim 50, wherein the endogenous antigen comprises a tumour-associated antigen (TAA) or a tumour-specific antigen (TSA).

52. The method or the agent for use according to any of claims 37 to 51, wherein the subject is a cancer patient or is at risk of developing a cancer.

53. The method or the agent for use according to any one of claims 37 to 49, wherein the antigen is an exogenous antigen.

54. The method or the agent for use according to claim 53, wherein the exogenous antigen comprises a viral, bacterial or fungal antigen.

55. The method or the agent for use according to claims 53 or 54, wherein the subject has an infectious disease.

56. The method or the agent for use according to any of claims 37 to 55, wherein the conjugate is not linked to a fluorescent protein.

57. The method or the agent for use according to any of claims 37 to 56, wherein the method is an in vivo method comprising administering the agent to the subject.

58. The method or the agent for use according to any of claims 37 to 57, wherein the agent is a vaccine.

59. The method or the agent for use according to claim 58, wherein the vaccine is administered in combination with an adjuvant.

60. The method or the agent for use according to claim 59, wherein the adjuvant is Alum.

61. The method or the agent for use according to any of claims 57 to 60, wherein the agent is administered in a solution comprising a pharmaceutically acceptable carrier.

62. The method or the agent for use according to any of claims 37 to 57, wherein the method is an ex vivo method comprising delivering the agent to the receiver cel in vitro.

63. The method or the agent for use according to claim 62, wherein the method further comprises contacting the receiver cel with the APC in vitro.

64. The method or the agent for use according to claim 63, wherein the method further comprises administering the APC to a subject in need thereof.

65. The method or the agent for use according to claim 63 or 64, wherein the APC had been obtained from the subject.

66. The method or the agent for use according to claim 63 or 65, wherein the method further comprises priming the lymphocyte in vitro and then administering the primed lymphocyte to the subject.

67. The method or the agent for use according to claim 66, wherein the lymphocyte had been obtained from the subject.


SEQUENCE LISTING (12 sequences)

SEQ ID NO: 1 - protein, 103 aa, synthetic construct
  VARIANT 47..55: note = X can be any amino acid
  VARIANT 81..89: note = X can be any amino acid
ATGVRAVPGNENSLEIEELARFAVDEHNKKENALLEFVRVVKAKEQXXXXXXXXXTMYYLTLEAKDGGKKKLYEAKVWVKXXXXXXXXXNFKELQEFKPVGDA

SEQ ID NO: 2 - protein, 103 aa, synthetic construct
ATGVRAVPGNENSLEIEELARFAVDEHNKKENALLEFVRVVKAKEQIDLTEWQDRTMYYLTLEAKDGGKKKLYEAKVWVKPEPIHSHHSNFKELQEFKPVGDA

SEQ ID NO: 3 - protein, 103 aa, synthetic construct
ATGVRAVPGNENSLEIEELARFAVDEHNKKENALLEFVRVVKAKEQWFDDEYDWITMYYLTLEAKDGGKKKLYEAKVWVKDYAATDLYWNFKELQEFKPVGDA

SEQ ID NO: 4 - protein, 103 aa, synthetic construct
ATGVRAVPGNENSLEIEELARFAVDEHNKKENALLEFVRVVKAKEQWEDFQTHWETMYYLTLEAKDGGKKKLYEAKVWVKDVGQLLSGINFKELQEFKPVGDA

SEQ ID NO: 5 - protein, 9 aa, synthetic construct
IDLTEWQDR

SEQ ID NO: 6 - protein, 9 aa, synthetic construct
PEPIHSHHS

SEQ ID NO: 7 - protein, 9 aa, synthetic construct
WFDDEYDWI

SEQ ID NO: 8 - protein, 9 aa, synthetic construct
DYAATDLYW

SEQ ID NO: 9 - protein, 9 aa, synthetic construct
WEDFQTHWE

SEQ ID NO: 10 - protein, 9 aa, synthetic construct
DVGQLLSGI

SEQ ID NO: 11 - protein, 10 aa, synthetic construct
GGGGSGGGGS

SEQ ID NO: 12 - protein, 15 aa, synthetic construct
ARTGGGGSGGGGSDI